(19) United States
(12) Patent Application Publication
(10) Pub. No.: US 2002/0082193 A1
(43) Pub. Date: Jun. 27, 2002

(54) Inhibition of mitochondrial calcium/sodium antiporter

(75) Inventors: Christen M. Anderson, Encinitas, CA (US); Robert E. Davis, San Diego, CA (US); Yazhong Pei, San Diego, CA (US); Soumitra S. Ghosh, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: MitoKor, San Diego, CA

(21) Appl. No.: 09/960,612

(22) Filed: Sep. 20, 2001

Related U.S. Application Data
(60) Provisional application No. 60/233,925, filed on Sep. 20, 2000.
(60) Provisional application No. 60/256,001, filed on Dec. 15, 2000.

(51) Int. Cl. A61K 31/00

(52) U.S. Cl. 514/1

(57) ABSTRACT

The invention provides compositions and methods for altering insulin secretion using an agent that inhibits calcium efflux via the mitochondrial calcium/sodium antiporter (MCA). Methods of treatment are thereby provided, and are particularly useful for treatment of subjects having, or suspected of being at risk for having, diabetes mellitus. Compositions and methods related to the identification of gene sequences encoding the mitochondrial calcium/sodium antiporter, expression of such sequences and screening assays using expressed MCA products are also provided.

### CROSS-REFERENCE TO RELATED APPLICATIONS

This application claims the benefit of U.S. Provisional Patent Application No. 60/233,925 filed Sep. 20, 2000; and U.S. Provisional Patent Application No. 60/256,001 filed Dec. 15, 2000, where these two provisional applications are incorporated herein by reference in their entireties.

### TECHNICAL FIELD

The invention relates generally to compositions and methods for altering insulin secretion using agents that affect mitochondrial activity. More specifically, the invention is directed to treatment methods involving the administration of an agent that alters mitochondrial regulation of intracellular calcium, in particular, by inhibiting calcium efflux via the mitochondrial calcium/sodium antiporter.

### BACKGROUND OF THE INVENTION

Type 2 diabetes mellitus, or “late onset” diabetes, is a common, degenerative disease affecting 5 to 10 percent of the population in developed countries. The propensity for developing type 2 diabetes mellitus (“type 2 DM”) is reportedly maternally inherited, suggesting a mitochondrial genetic involvement. (Alcolado, J. C. and Alcolado, R., Br. Med. J. 302:1178-1180 (1991); Reny, S. L., International J. Epidem. 23:886-890 (1994)). Diabetes is a heterogeneous disorder with a strong genetic component; monozygotic twins are highly concordant and there is a high incidence of the disease among first degree relatives of affected individuals.

At the cellular level, the pathologic phenotype that may be characteristic of the presence of, or risk for predisposition to, late onset diabetes mellitus includes the presence of one or more indicators of altered mitochondrial respiratory function, for example impaired insulin secretion, decreased ATP synthesis and increased levels of reactive oxygen species. Studies have shown that type 2 DM may be preceded by or associated with certain related disorders. For example, it is estimated that forty million individuals in the U.S. suffer from impaired glucose tolerance (IGT). Following a glucose load, circulating glucose concentrations in IGT patients rise to higher levels, and return to baseline levels more slowly, than in unaffected individuals. A small percentage of IGT individuals (5-10%) progress to non-insulin dependent diabetes (NIDDM) each year. This form of diabetes mellitus, type 2 DM, is associated with decreased release of insulin by pancreatic beta cells and a decreased end-organ response to insulin. Other symptoms of diabetes mellitus and conditions that precede or are associated with diabetes mellitus include obesity, vascular pathologies, peripheral and sensory neuropathies and blindness.

Glucose-mediated insulin secretion from the pancreatic beta cell is triggered by a complex sequence of intracellular events (FIG. 1). Glucose is taken up by the beta cell via GLUT-2 glucose transporters; it is subsequently phosphorylated by glucokinase to glucose-6-phosphate, which enters the glycolytic pathway. The reducing equivalents (NADH) and substrate (pyruvate) produced through glycolysis enter the mitochondria and fuel increased respiration and oxidative phosphorylation. The consequent rise in cellular ATP levels triggers closure of the K+-ATP channels at the plasma membrane, depolarizing the membrane and permitting influx of calcium. Calcium appears to have two main roles: stimulating release of insulin from the cells (e.g., Kennedy et al., 1996 J. Clin. Invest. 98:2524; Maechler et al., 1997 EMBO J. 16:3833), and acting as a “feed-forward” regulator of mitochondrial ATP production (e.g., Cox and Matlib, 1993 Trends Pharmacol. Sci. 14:408). The latter is accomplished by mitochondrial uptake of calcium through the mitochondrial calcium uniporter (e.g., Newgard et al., 1995 Ann. Rev. Biochem. 64:689; Magnus et al., 1998 Am. J. Physiol. 274:C1174-C1184). The rise in mitochondrial calcium stimulates respiration and oxidative phosphorylation through stimulation of calcium-sensitive dehydrogenase (Rutter et al., 1988 Biochem. J. 252:181; Rutter et al., 1993 J. Biol. Chem. 268:22385). However, the rise in mitochondrial calcium is transient, since calcium returns to the cytoplasm through regulated calcium efflux channels, for instance a mitochondrial calcium antiporter such as the mitochondrial calcium/sodium antiporter (MCA) also known as the mitochondrial sodium/calcium exchanger (mNCE; see, e.g., Newgard 1995; Magnus 1998; for a general review of mitochondrial membrane transporters, see, e.g., Zonatti et al., 1994 J. Bioenergetics Biomembr. 26:543 and references cited therein). The use of MCA inhibitors has been contemplated for their potential effects on cardiac function (e.g., Cox and Matlib, 1993 Trends Pharmacol. Sci. 14:408-413), but such use has not been suggested for certain other indications such as diabetes. Thus, for example, while elevated intramitochondrial calcium concentration has been correlated with insulin secretion and oxidative ATP synthesis, as noted above (e.g., Kennedy et al., 1996 J. Clin. Invest. 98:2524; Maechler et al., 1997 EMBO J. 16:3833; Cox and Matlib, 1993 Trends Pharmacol. Sci. 14:408), no inducer-effector relationship between oxidative ATP synthesis and insulin secretion has been universally accepted (see, e.g., Newgard, 1995 Ann. Rev. Biochem. 64:689). Moreover, currently available inhibitors of the MCA are regarded as either not specific for the MCA, or useful only at extremely high concentrations, precluding their apparent suitability for pharmaceutical compositions (Cox and Matlib, 1993 Trends Pharmacol. Sci. 14:408-413).

Current pharmacological therapies for type 2 DM include injected insulin, and oral agents that are designed to lower blood glucose levels. Currently available oral agents include (i) the sulfonylureas, which act by enhancing the sensitivity of the pancreatic beta cell to glucose, thereby increasing insulin secretion in response to a given glucose load; (ii) the biguanides, which improve glucose disposal rates and inhibit hepatic glucose output; (iii) the thiazolidinediones, which improve peripheral insulin sensitivity through interaction with nuclear peroxisome proliferator-activated receptors (PPAR, see, e.g., Spiegelman, 1998 Diabetes 47:507-514; Schoonjans et al., 1997 Curr. Opin. Lipidol. 8:159-166; Staels et al., 1997 Biochimie 79:95-99), (iv) repaglinide, which enhances insulin secretion through interaction with ATP-dependent potassium channels; and (v) acarbose, which decreases intestinal absorption of carbohydrates. Although currently available drugs for treating type 2 diabetes, such as the sulfonylureas, improve insulin secretion, both basal and insulin stimulated insulin secretion are enhanced by such compounds. Consequently, undesirable chronic hyperinsulinemia, hypoglycemia and/or excessive weight gain may result following treatment with such drugs (Cobb et al., 1998 Ann. Rep. Med. Chem. 33:213-222; Krentz et al., 1994 Drug Safety 11:223-241).

It is therefore clear that none of the current pharmacological therapies corrects the underlying biochemical defect in type 2 DM. Neither do any of these currently available treatments improve all of the physiological abnormalities in type 2 DM such as impaired insulin secretion, insulin resistance and/or excessive hepatic glucose output. In addition, treatment failures are common with these agents, such that multi-drug therapy is frequently necessary.

Mitochondria are organelles that are the main energy source in cells of higher organisms. These organells provide direct and indirect biochemical regulation of a wide array of cellular respiratory, oxidative and metabolic processes, including metabolic energy production, aerobic respiration and intracellular calcium regulation. For example, mitochondria are the site of electron transport chain (ETC) activity, which drives oxidative phosphorylation to produce metabolic energy in the form of adenosine triphosphate (ATP), and which also underlies a central mitochondrial role in intracellular calcium homeostasis. These processes require the maintenance of a mitochondrial membrane electrochemical potential, and defects in such membrane potential can result in a variety of disorders.

Mitochondria contain an outer mitochondrial membrane that serves as an interface between the organelle and the cytosol, a highly folded inner mitochondrial membrane that appears to form attachments to the outer membrane at multiple sites, and an intermembrane space between the two mitochondrial membranes. The subcompartment within the inner mitochondrial membrane is commonly referred to as the mitochondrial matrix (for review, see, e.g., Emster et al., J. Cell Biol. 91:227s, 1981). While the outer membrane is freely permeable to ionic and non-ionic solutes having molecular weights less than about ten kilodaltons, the inner mitochondrial membrane exhibits selective and regulated permeability for many small molecules, including certain cations, and is impermeable to large (greater than about 10 kD) molecules.

Four of the five multisubunit protein complexes (Complexes I, III, IV and V) that mediate ETC activity are localized to the inner mitochondrial membrane. The remaining ETC complex (Complex II) is situated in the matrix. In at least three distinct chemical reactions known to take place within the ETC, protons are moved from the mitochondrial matrix, across the inner membrane, to the intermembrane space. This disequilibrium of charged species creates an electrochemical membrane potential of approximately 220 mV referred to as the “protonmotive force” (PMF). The PMF, which is often represented by the notation Δp, corresponds to the sum of the electric potential (ΔΨm) and the pH differential (ΔpH) across the inner membrane according to the equation

Δp=ΔΨm−ZΔpH

wherein Z stands for −2.303 RT/F. The value of Z is −59 at 25° C. when Δp and ΔΨm are expressed in mV and ΔpH is expressed in pH units (see, e.g., Emster et al., J. Cell Biol. 91 :227s, 1981, and references cited therein).

ΔΨm provides the energy for phosphorylation of adenosine diphosphate (ADP) to yield ATP by ETC Complex V, a process that is coupled stoichiometrically with transport of a proton into the matrix. ΔΨm is also the driving force for the influx of cytosolic Ca²⁺ into the mitochondrion. Normal alterations of intramitochondrial Ca²⁺ are associated with normal metabolic regulation (Dykens, 1998 in Mitochondria & Free Radicals in Neurodegenerative Diseases, Beal, Howell and Bodis-Wollner, Eds., Wiley-Liss, New York, pp. 29-55; Radi et al., 1998 in Mitochondria & Free Radicals in Neurodegenerative Diseases, Beal, Howell and Bodis-Wollner, Eds., Wiley-Liss, New York, pp. 57-89; Gunter and Pfeiffer, 1991, Am. J. Physiol. 27: C755; Gunter et al., Am. J. Physiol. 267:313, 1994). For example, fluctuating levels of mitochondrial free Ca²⁺ may be responsible for regulating oxidative metabolism in response to increased ATP utilization, via allosteric regulation of enzymes (reviewed by Crompton and Andreeva, Basic Res. Cardiol. 88:513-523, 1993); and the glycerophosphate shuttle (Gunter and Gunter, J. Bioenerg. Biomembr. 26:471, 1994).

Normal mitochondrial function includes regulation of cytosolic free calcium levels by sequestration of excess Ca²⁺ within the mitochondrial matrix, including transiently elevated cytosolic free calcium that results from physiologic biological signal transduction. Depending on cell type, cytosolic Ca²⁺ concentration is typically 50-100 nM. In normally functioning cells, when Ca²⁺ levels reach 200-300 nM, mitochondria begin to accumulate Ca²⁺ as a function of the equilibrium between influx via a Ca²⁺ uniporter in the inner mitochondrial membrane and Ca²⁺ efflux via both Na⁺ dependent and Na⁺ independent calcium carriers, including notably the MCA. The low affinity of this rapid uniporter mechanism suggests that the primary uniporter function may be to lower cytosolic Ca²⁺ in response to elevation of cytosolic free calcium levels, which may result from calcium influx across the plasma membrane that occurs as part of a biological signal transduction mechanism (Gunter and Gunter, J. Bioenerg. Biomembr. 26:471, 1994; Gunter et al., Am. J. Physiol. 267:313, 1994). In certain instances, for example in pancreatic beta cells, physiologic rises in cytoplasmic calcium occur in response to glucose (or other secretagogues) and lead to calcium uptake by mitochondria, stimulating increased ATP synthesis. Similarly, the primary calcium antiporter (e.g., MCA) function may be to lower mitochondrial Ca²⁺ concentrations in response to mitochondrial Ca²⁺ influxes, such as may result from glucose stimulation of a glucose-sensitive cell, and which produce transient increases in oxidative ATP synthesis. Thus, mitochondrially regulated calcium cycling between, inter alia, cytosolic and mitochondrial compartments may provide an opportunity for manipulation of intracellular ATP levels (e.g., Cox and Matlib, 1993 Trends Pharmacol. Sci. 14:408-413; Matlib et al., 1983 Eur. J. Pharmacol. 89:327; Matlib 1985 J. Pharmacol. Exp. Therap. 233:376; Matlib et al. 1983 Life Sci. 32:2837).

In view of the significance of mitochondrial regulation of intracellular calcium and the relationship of this mitochondrial activity to diabetes, which includes any of a wide range of disease states characterized by inappropriate and sustained hyperglycemia, there is clearly a need for improved compositions and methods to control mitochondrial calcium homeostasis. To provide improved therapies for diabetes, agents that alter mitochondrial calcium cycling between intramitochondrial and extramitochondrial subcellular compartments may be beneficial, and assays to specifically detect such agents are needed. Clearly, for example, there is a need for improved therapeutics that are targeted to correct biochemical and/or metabolic defects responsible for, or associated with, type 2 DM, regardless of whether such a defect underlying altered mitochondrial function may have mitochondrial or extramitochondrial origins. The present invention provides compositions and methods related to modulation of mitochondrial calcium/sodium antiporter function that are useful for treating diabetes, and in particular, type 2 DM, by enhancing insulin secretion, and offers other related advantages.

### Summary of the Invention

The present invention is directed in part to a method for treating diabetes mellitus, comprising: administering, to a subject having or suspected of being at risk for having diabetes mellitus, a therapeutically effective amount of a pharmaceutical composition comprising an agent that selectively impairs a mitochondrial calcium/sodium antiporter activity. In certain embodiments the agent enhances insulin secretion and in certain other embodiments the agent enhances insulin secretion that is stimulated by glucose. In certain other embodiments the agent enhances insulin secretion that is stimulated by a supraphysiological glucose concentration and does not enhance insulin secretion in the presence of a physiological glucose concentration. In certain further embodiments the method further comprises administering to the subject one or more agent that lowers circulating glucose concentration in the subject, which agent in certain still further embodiments is insulin, an insulin secretagogue, an insulin sensitizer, an inhibitor of hepatic glucose output or an agent that impairs glucose absorption. In certain other further embodiments the insulin secretagogue is a sulfonylurea compound or a nonsulfonylurea compound (e.g., repaglinide).

In certain embodiments the diabetes mellitus is type 2 diabetes mellitus or maturity onset diabetes of the young. In certain embodiments the pharmaceutical composition is administered orally. In certain embodiments the agent does not substantially alter insulin secretion in the presence of a physiological glucose concentration. In certain embodiments the candidate agent is membrane permeable. In certain embodiments the membrane is at least one of the membranes selected from the group consisting of a plasma membrane and a mitochondrial membrane. In certain embodiments the mitochondrial membrane is selected from the group consisting of an inner mitochondrial membrane and an outer mitochondrial membrane.

In certain embodiments there is provided a method for determining the presence of a mitochondrial calcium/sodium antiporter polypeptide in a biological sample comprising: contacting a biological sample containing a mitochondrial calcium/sodium antiporter polypeptide with a mitochondrial calcium/sodium antiporter ligand under conditions and for a time sufficient to allow binding of the mitochondrial calcium/sodium antiporter ligand to a mitochondrial calcium/sodium antiporter polypeptide; and detecting the binding of the mitochondrial calcium/sodium antiporter ligand to a mitochondrial calcium/sodium antiporter polypeptide, and therefrom determining the presence of a mitochondrial calcium/sodium antiporter polypeptide in said biological sample.

In certain embodiments the mitochondrial calcium/sodium antiporter ligand comprises a compound of structure (I), such as Compound No. 1, as defined below. In certain embodiments the mitochondrial calcium/sodium antiporter ligand is detectably labeled. In certain embodiments the detectably labeled mitochondrial calcium/sodium antiporter ligand comprises a radiolabeled substituent. In certain embodiments the radiolabeled substituent is selected from the group consisting of ¹²⁵I, ¹³¹I, ³H, ¹⁴C, ⁴⁵Ca and ³⁵S. In certain embodiments the detectably labeled mitochondrial calcium/sodium antiporter ligand comprises a fluorescent substituent. In certain embodiments the detectably labeled mitochondrial calcium/sodium antiporter ligand comprises covalently bound biotin.

In certain embodiments there is provided a method for isolating a mitochondrial calcium/sodium antiporter from a biological sample, comprising: contacting a biological sample suspected of containing a mitochondrial calcium/sodium antiporter polypeptide with a mitochondrial calcium/sodium antiporter ligand under conditions and for a time sufficient to allow binding of the mitochondrial calcium/sodium antiporter ligand to a mitochondrial calcium/sodium antiporter polypeptide; and recovering the mitochondrial calcium/sodium antiporter polypeptide, and thereby isolating a mitochondrial calcium/sodium antiporter from a biological sample. In certain embodiments the mitochondrial calcium/sodium antiporter ligand is covalently bound to a solid phase. In certain embodiments the mitochondrial calcium/sodium antiporter ligand is non-covalently bound to a solid phase.

These and other aspects of the present invention will become apparent upon reference to the following detailed description and attached drawings. All references disclosed herein are hereby incorporated by reference in their entireties as if each was incorporated individually.

### BRIEF DESCRIPTION OF THE DRAWINGS

FIG. 1 shows a model of glucose-mediated insulin secretion in pancreatic beta cells.

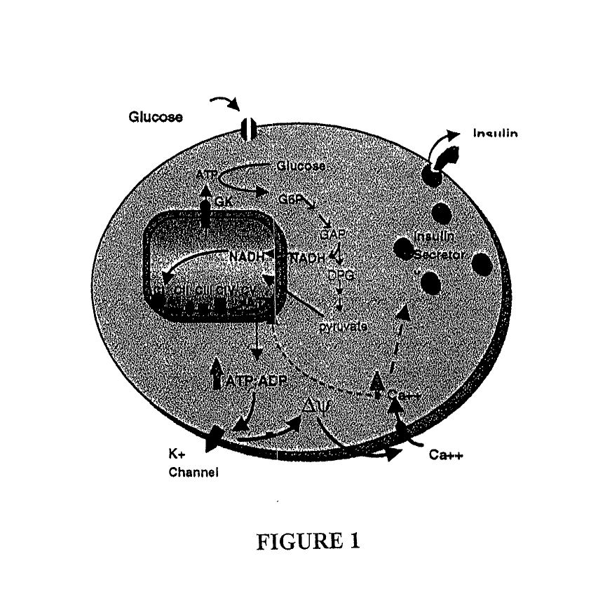

FIG. 2 shows enhancement of glucose stimulated insulin secretion by INS-1 cells exposed to Compound No. 1.

FIG. 3 shows enhancement of glucose stimulated insulin secretion by rat islets exposed to Compound No. 1.

FIG. 4 shows inhibition of MCA activity in rat heart mitochondria by Compound No. 5, detected with a calcium electrode.

FIG. 5 shows inhibition of MCA activity in mitochondria by Compound No. 4, detected by Calcium Green SN fluorescence.

FIG. 6 shows insulin secretion by rat islets exposed to Compound No. 1 alone and in combination with other secretagogues.

FIG. 7 shows affinity isolation of a mitochondrial calcium/sodium antiporter using immobilized Compound No. 5. Lanes 1 and 9, molecular weight markers; lane 2, beef heart mitochondria total protein extract; lane 3, Compound No. 5-column-passed material; lane 4, 25 mM TEA/TES wash; lane 5, 100 mM TEA/TES wash; lane 6, 10 mM TPP eluate; lane 7, 1 M NaCl wash; lane 8, 10 mM Cpd 1/40% PEG 400/10% EtOH eluate.

FIG. 8 shows inhibition by Compound No. 1 of sodium-calcium exchange in proteoliposomes reconstituted with MCA activity affinity isolated on immobilized Compound No. 5.

### DETAILED DESCRIPTION OF THE INVENTION

The present invention is directed in part to a method for treating diabetes mellitus, by administering to a subject having or suspected of being at risk for having diabetes a pharmaceutical composition that contains a selective inhibitor of the mitochondrial calcium/sodium antiporter (MCA). As described in greater detail herein, such an inhibitor substantially enhances insulin secretion in preferred embodiments, and in certain preferred embodiments the MCA inhibitor enhances insulin secretion that is stimulated by supraphysiological glucose concentrations (e.g., glucose stimulated insulin secretion), but does not substantially enhance insulin secretion under conditions where normal physiological glucose concentrations are present (e.g., basal insulin secretion). The invention therefore relates in part to the unexpected observation that diabetes may be effectively treated using certain agents, wherein such agents may be selected that interfere with MCA and/or other mitochondrial calcium efflux mechanisms in a manner that preferentially enhances glucose stimulated insulin secretion relative to basal insulin secretion. Thus, as elaborated upon below, the present invention provides heretofore unrecognized advantages associated with impairment of MCA activity, as such advantages pertain to treatment of diabetes.

According to non-limiting theory, the present invention relates to a method that exploits pharmacological intervention to maintain increased and sustained intramitochondrial calcium concentrations, thereby driving oxidative phosphorylation and the consequent elevation of intracellular ATP concentration. Further according to this theory, such elevated ATP concentrations promote enhanced insulin secretion as provided herein and effect the desirable result of providing sufficient insulin to lower supraphysiological circulating glucose concentrations and preferably return them to concentrations at or near normal levels.

In certain other preferred embodiments of the present invention, there is provided a method for treating diabetes comprising administering to a subject a therapeutically effective amount of an agent that selectively impairs a MCA activity, as provided herein, and further comprising administering an agent that lowers circulating glucose concentrations. While current agents for treating type 2 DM may lower blood glucose levels without correcting underlying biochemical defects in this disease, as noted above, it may therefore be desirable in certain instances to combine an agent that impairs a MCA activity according to the instant disclosure with an existing hypoglycemic agent. Thus, for example by way of illustration and not limitation, a drug of the sulfonylurea class or of the more recently developed non-sulfonylurea class of agents that close the potassium/ATP channel may be combined with an agent that impairs a MCA activity. As other non-limiting example, agents that supply substrates for mitochondrial metabolism (e.g., KCl, α-ketoisocaproic acid or leucine), insulin sensitizers (e.g., thiazolidinediones), inhibitors of hepatic glucose output (e.g., metformin) or glucose uptake blockers (e.g., acarbose) may also enhance the effect of an agent that impairs a MCA activity in the treatment of type 2 DM or potentially other conditions.

In the context of this invention, an agent that selectively impairs a mitochondrial calcium/sodium antiportor activity includes a compound having the following general structure (I):

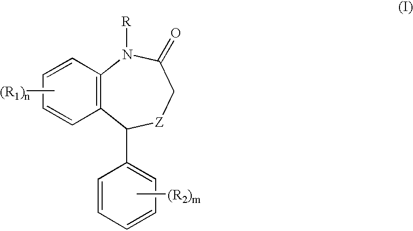

including stereoisomers, prodrugs and pharmaceutically acceptable salts thereof,

wherein

Z is O, S, S(═O) or S(═O)₂;

R is hydrogen, alkyl or substituted alkyl;

R₁ and R₂ are the same or different and at each occurrence are independently halogen, cyano, nitro, mono- or di-alkylamino, alkyl, substituted alkyl, aryl, substituted aryl, arylalkyl, substituted arylalkyl, heterocycle, substituted heterocycle, heterocyclealkyl or substituted heterocyclealkyl; and

n and m are the same or different, and independently 0, 1, 2, 3 or 4.

As used herein, the terms used above have the following meaning:

“Alkyl” means a straight chain or branched, saturated or unsaturated, cyclic or non-cyclic hydrocarbon having from 1 to 10 carbon atoms, while “lower alkyl” has the same meaning but only has from 1 to 6 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Unsaturated alkyls contain at least one double or triple bond between adjacent carbon atoms (also referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1 butynyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like. Cycloalkyls are also referred to herein as “carbocyclic” rings systems, and include bi- and tri-cyclic ring systems having from 8 to 14 carbon atoms such as a cycloalkyl (such as cyclo pentane or cyclohexane) fused to one or more aromatic (such as phenyl) or non-aromatic (such as cyclohexane) carbocyclic rings.

“Halogen” means fluorine, chlorine, bromine or iodine.

“Oxo” means a carbonyl group (i.e., ═O).

“Cyano” means—CN.

“Nitro” means—NO₂.

“Haloalkyl” means an alkyl having at least one hydrogen atom replace with halogen, such as trifluoromethyl and the like.

“Mono- or di-alkylamino means an amino (i.e., —NH₂) having one hydrogen atom replaced with an alkyl or having both hydrogen atoms replaced with an alkyl, respectively.

“Alkanediyl” means a divalent alkyl from which two hydrogen atoms are taken from the same carbon atom or from different carbon atoms, such as —CH₂——CH₂CH₂—, —CH₂CH₂CH₂—, —CH(CH₃)CH₂—, and the like.

“Aryl” means an aromatic carbocyclic moiety such as phenyl or naphthyl.

“Arylalkyl” means an alkyl having at least one alkyl hydrogen atom replaced with an aryl moiety, such as benzyl, —(CH₂)₂phenyl, —(CH₂)₃phenyl, —CH(phenyl)₂, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, and quinazolinyl.

“Heteroarylalkyl” means an alkyl having at least one alkyl hydrogen atom replaced with a heteroaryl moiety, such as —CH₂pyridinyl, —CH₂pyrimidinyl, and the like.

“Heterocycle” means a 5- to 7-membered monocyclic, or 7- to 10-membered bicyclic, heterocyclic ring which is either saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined above. Thus, in addition to the heteroaryls listed above, heterocycles also include morpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocyclealkyl” means an alkyl having at least one alkyl hydrogen atom replaced with a heterocycle, such as —CH₂ morpholinyl, and the like.

The term “substituted” as used herein means any of the above groups (i.e., alkyl, aryl, arylalkyl, heteroaryl, heteroarylalkyl, heterocycle and/or heterocyclealkyl) wherein at least one hydrogen atom is replaced with a substituent. In the case of an oxo substituent (“═O”) two hydrogen atoms are replaced. Substituents include halogen, hydroxy, alkyl, substituted alkyl (such as haloalkyl, mono- or di-substituted aminoalkyl, alkyloxyalkyl, and the like), aryl, substituted aryl, arylalkyl, substituted arylalkyl, heterocycle, substituted heterocycle, heterocyclealkyl, substituted heterocyclealkyl, —NRₐR_{b}, —NRₐC(═O)R_{b}, —NR_{c}C(═O)NRₐR_{b}, —NRₐC(═O)OR_{b}—NRₐSO₂R_{b}, —ORₐ, —C(═O)Rₐ—C(═O)ORₐ—C(═O)NRₐR_{b}, —OC(═O)Rₐ, —OC(═O)ORₐ, —OC(═O)NRₐR_{b}, —NRₐSO₂R_{b}, —CONRₐ{alkanediyl)OR_{b}, —CONR_{c}{alkanediyl-O)₁₋₆(alkanediyl)NRₐR_{b}, or a radical of the formula —Y—Z—Rₐ where Y is alkanediyl, substituted alkanediyl, or a direct bond, Z is —O—, —S—, —S(═O)—, —S(═O)₂—, —N(R_{b})—, —C(═O)—, —C(═O)O—, —OC(═O)—, —N(R_{b})C(═O)—, —C(═O)N(R_{b})— or a direct bond, wherein Rₐ, R_{b} and R_{c} are the same or different and independently hydrogen, amino, alkyl, substituted alkyl (including haloalkyl), aryl, substituted aryl, arylalkyl, substituted arylalkyl, heterocycle, substituted heterocycle, heterocylealkyl or substituted heterocyclealkyl, or wherein Rₐ and R_{b} taken together with the nitrogen atom to which they are attached form a heterocycle or substituted heterocycle.

In one embodiment Z is sulfur, and in another embodiment Z is oxygen, and the compound has the following structure (II) or (III), respectively:

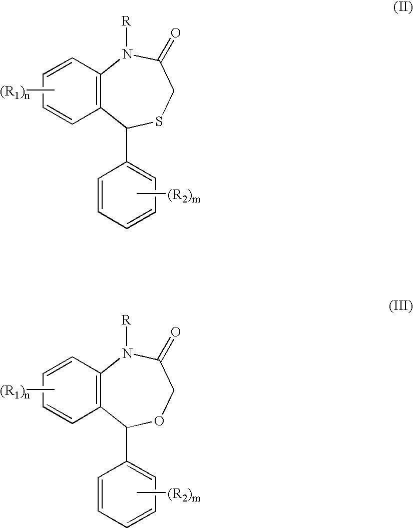

In more specific embodiments, R of structures (II) and (III) is hydrogen, and the compound has the following structure (II-1) or (III-1)

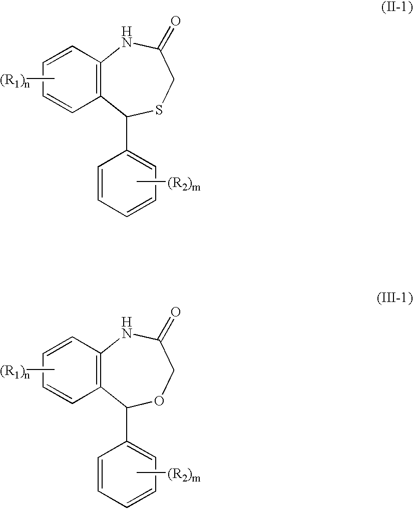

In still more specific embodiments, n and m are both 1, R₁ and R₂ of structure (II-1) and (III-1) are both halogen, and the compound has the following structure (II-2) or (III-2), wherein each occurrence of “X” is the same or different and independently selected from a halogen (i.e., fluoro, chloro, bromo or iodo):

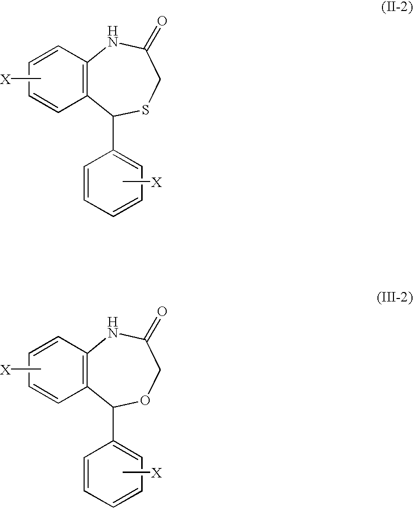

In a further embodiment, R is a substituted alkyl, such as methyl, wherein the substituent is —C(═O)ORₐ, and the compound has the following structure (IV):

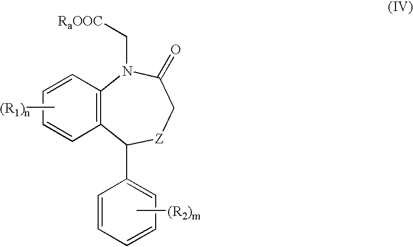

In more specific embodiments of structure (IV), Rₐ is hydrogen or alkyl, such as methyl, ethyl, n-propyl, i-propyl, n-butyl, i-butyl, t-butyl and the like.

In other embodiments, R is again a substituted alkyl, such as methyl, but substituted with —CONRₐ{alkanediyl)OR_{b} or —CONR_{c}{alkanediyl-O)₁₋₆(alkanediyl)NRₐR_{b}, as represented by structures (V) and (VI), respectively:

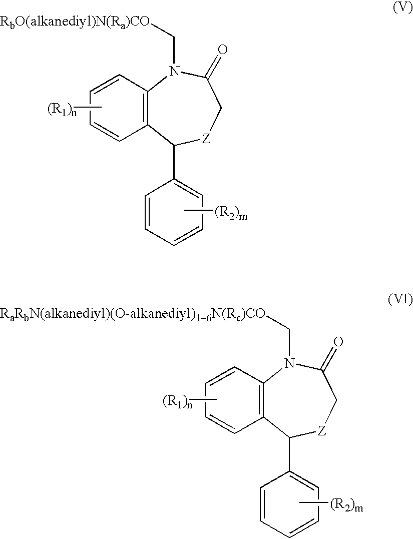

With regard to location of the R₁ and R₂ groups, the following numbering scheme is used herein:

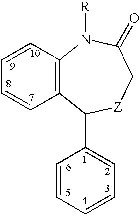

Thus, in more specific embodiments, n and m are both 1, R₁ is at the 8-position and R₂ is at the 2-position, and the compound has the following structure (VII):

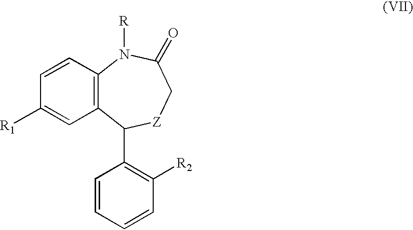

The compounds of structure (I) above may be made by techniques knows to those skilled in the field of organic chemistry, and as more specifically exemplified in the Examples. However, in general, such compounds may be made by the following reaction schemes.

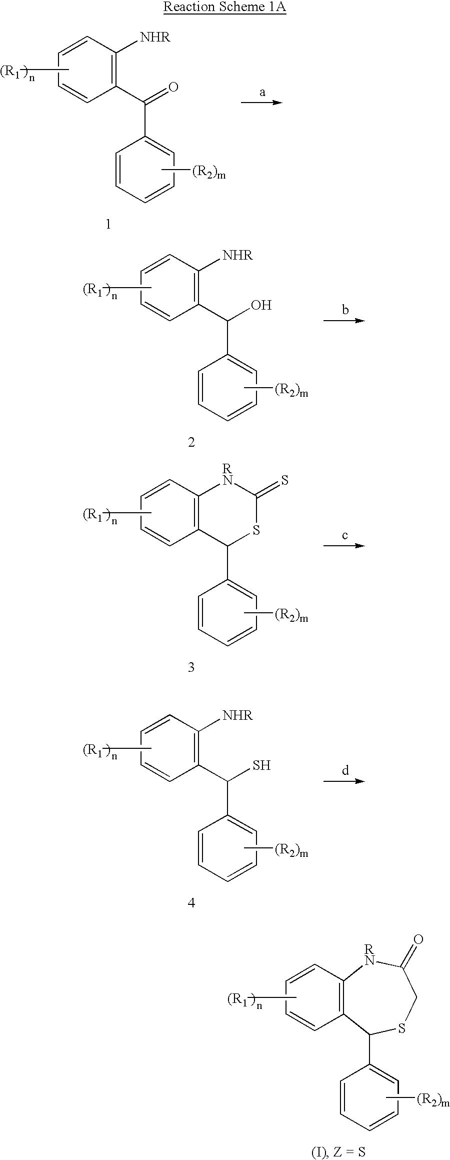

In step a of the above reaction scheme, ketone 1 is converted to the corresponding alcohol 2 by reaction with NaBH₄, which is then converted in step b to intermediate 3 by contact with CS₂. This intermediate is converted to thiol 4 in step c by reaction first with H₂O₂ and KOH, and second with Na₂S₂O₄ and NaOH. Compound of structure (I), wherein Z=S is formed in step d by reaction with ClCOCH₂Cl. The synthesis of such benzothiazepine systems is described in greater detail by Hirai et al. in U.S. Pat. Nos. 4,297,280 and 4,341,704.

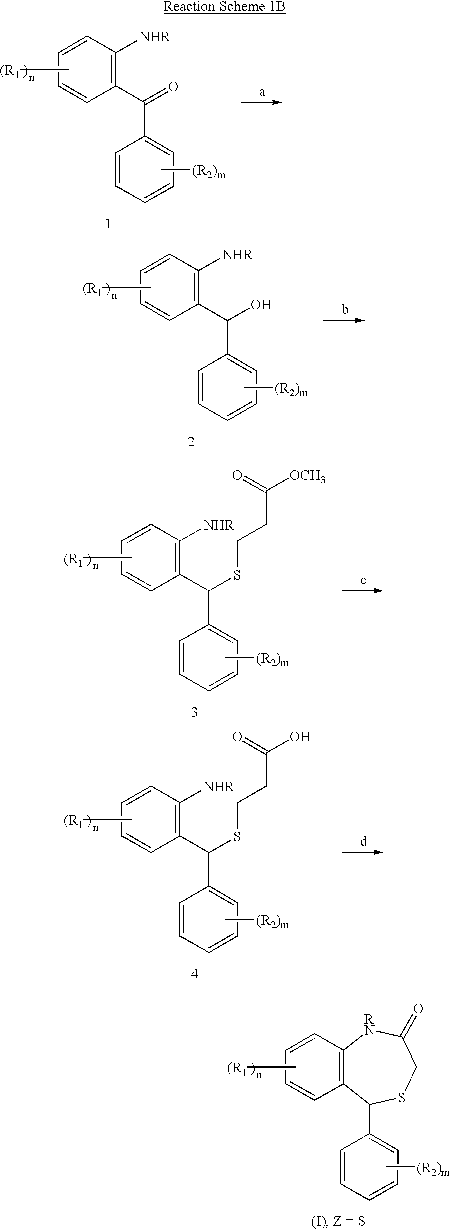

Alternatively, in step a of the above reaction scheme, starting ketone 1 is reduced to alcohol 2, followed by treatment with methylglycolate in the presence of TFA in step b to yield thioether 3. Alkaline hydrolysis is accomplished in step c, followed by cyclization in step d to provide the desired compound of structure (I) (where Z=S).

Conversion of the thioether (Z=S) to the corresponding sulfinyl (Z=SO) and sulfonyl (Z=SO₂) analogs may be achieved by oxidation with sodium periodate in aqueous THF solution at room temperature.

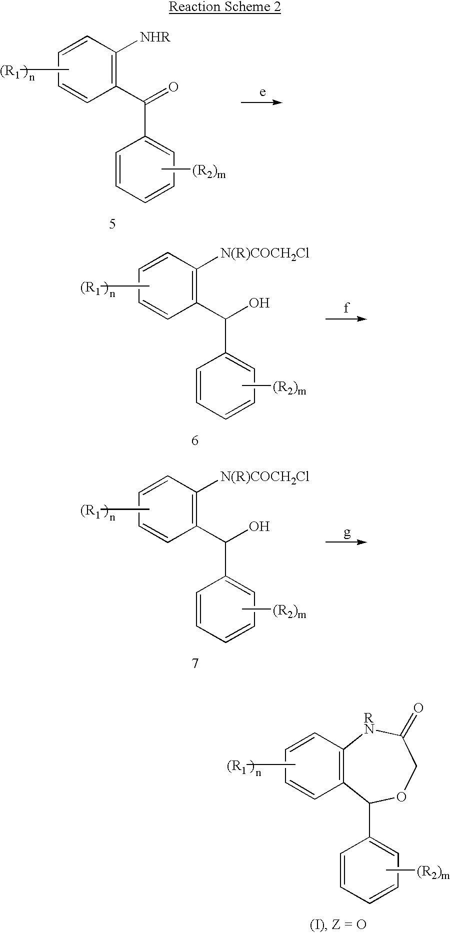

In step e of the above reaction scheme, the amine group of ketone 5 is converted to intermediate 6 by reaction with ClCH₂COCl, which is then converted in step f to the corresponding alcohol 7 by contact NaBH₄. Compound of structure (I), wherein Z=0 and R=H is formed in step g by reaction with Na/iPrOH. The synthesis of such benzoxazepine systems is described in greater detail by Hirai et al. in U.S. Pat. Nos. 4,297,280 and 4,341,704.

### Reaction Scheme 3

In Reaction Schemes 1A, 1B and 2 above, the R moiety may be hydrogen. In this case, the nitrogen group may be deprotonated using known techniques, followed by addition of the desired R group, as more fully described in the examples.

“Pharmaceutically acceptable salt” refers to salts of the compounds of the present invention derived from the combination of such compounds and an organic or inorganic acid (acid addition salts) or an organic or inorganic base (base addition salts). The compounds of the present invention may be used in either the free base or salt forms, with both forms being considered as being within the scope of the present invention.

The compounds of the present invention may generally be utilized as the free acid or base. Alternatively, the compounds of this invention may be used in the form of acid or based addition salts. Acid addition salts of the free base amino compounds of the present invention may be prepared by methods well known in the art, and may be formed from organic and inorganic acids. Suitable organic acids include maleic, fumaric, benzoic, ascorbic, succinic, methanesulfonic, acetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Based addition salts include the ammonium ion, as well as other suitable cations. Thus, the term “pharmaceutically acceptable salt” of structure (I) is intended to encompass any and all acceptable salt forms.

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound.

With regard to stereoisomers, the compounds of structure (I) may have chiral centers and may occur as racemates, racemic mixtures and as individual enantiomers or diastereomers. All such isomeric forms are included within the present invention, including mixtures thereof. Furthermore, some of the crystalline forms of the compounds of structure (I) may exist as polymorphs, which are included in the present invention. In addition, some of the compounds of structure (I) may also form solvates with water or other organic solvents. Such solvates are similarly included within the scope of this invention.

An “agent that lowers circulating glucose concentrations” includes any hypoglycemic agent as known in the art and provided herein, including anti-diabetic agents such as sulfonylurea compounds and non-sulfonylurea compounds, and may further include a biguanide, a thiazolidinedione, repaglinide, acarbose, metformin or other hypoglycemic compositions (e.g., 6LP-1 and its analogs, DPP-IV inhibitors, α-ketoisocaproic acid, leucine or analogs of other amino acids).

A “biological sample” may comprise any tissue or cell preparation as described herein and a “biological sample containing a mitochondrial calcium/sodium antiporter polypeptide” comprises any tissue or cell preparation in which an expressed MCA polypeptide or other mitochondrial molecular component as provided herein that mediates Ca²⁺ efflux from a mitochondrion is thought to be present. Biological samples (including those containing a MCA polypeptide) may be provided by obtaining a blood sample, biopsy specimen, tissue explant, organ culture or any other tissue or cell preparation from a subject or a biological source. The subject or biological source may be a human or non-human animal, a primary cell culture or culture adapted cell line including but not limited to genetically engineered cell lines that may contain chromosomally integrated or episomal recombinant nucleic acid sequences, immortalized or immortalizable cell lines, somatic cell hybrid or cytoplasmic hybrid “cybrid” cell lines, differentiated or differentiatable cell lines, transformed cell lines and the like. A biological sample may, for example, be derived from a recombinant cell line or from a transgenic animal.

In certain preferred embodiments the subject or biological source is a human known to have, or suspected of being at risk for having, diabetes mellitus. In certain further preferred embodiments the diabetes mellitus is type 2 diabetes mellitus, and in certain other further preferred embodiments the diabetes mellitus is maturity onset diabetes of the young (MODY). Well known criteria have been established for determining a presence of, or risk for having diabetes mellitus (e.g., type 2 diabetes mellitus, MODY) as described herein and as known in the art, and these may be found, for example, in Clinical Practice Recommendations 2000 (2000 Diabetes Care 23:supplement 1) or elsewhere (see, e.g., www.diabetes.org/, the website of the American Diabetes Association). Among these recognized physiological parameters that relate to diabetes, those familiar with the art will appreciate that a variety of methodologies have been established for the determination of glucose and insulin concentrations in the circulation. For example, methods for quantifying insulin in a biological sample as provided herein (e.g., a blood, serum or plasma sample) may include a radioimmunoassay (RIA) using an antibody that specifically binds to insulin. Variations on RIA such as enzyme linked immunosorbent assays and immunoprecipitation analysis, and other assays for the presence of insulin or proinsulin in a biological sample are readily apparent to those familiar with the art, and may further include assays that measure insulin secretion by cells in the presence or absence of secretagogues such as glucose, KCl, amino acids, sulfonylureas, forskolin, glyceraldehyde, succinate or other agents that may increase or decrease insulin or proinsulin in a cell conditioned medium. Such methods may also be used to quantify the amount of insulin produced by or released from an insulin-secreting cell.

Because it is well recognized by those familiar with the art that there may be large quantitative variations in circulating glucose and insulin levels among individual subjects (see, e.g., Clinical Practice Recommendations 2000, 2000 Diabetes Care 23 (suppl. 1), and references cited therein), the present invention contemplates in preferred embodiments a method for treating diabetes with a pharmaceutical composition comprising an agent that selectively impairs MCA activity as provided herein, wherein the agent does not substantially enhance insulin secretion at physiological glucose concentration (i.e., under fasting or basal metabolic conditions) and wherein the agent substantially enhances insulin secretion at supraphysiological glucose concentration (i.e., under non-fasting conditions or conditions of glucose stimulation). Although certain preferred embodiments of the present invention relate to compositions and methods for treating diabetes in humans, the invention need not be so limited. In particular, those having ordinary skill in the art will readily appreciate that diabetes, including any disease state characterized by inappropriate and/or sustained periods of hyperglycemia such as type 2 DM or other diabetes mellitus, may be a condition that is present in a number of non-human animals (e.g., Ford, 1995 Veterin. Clinics of N. Amer.: Small Animal Practice 25(3):599-615). Accordingly, compositions and methods provided herein as may be useful for the treatment of these and other manifestations of diabetes in non-human animals are within the scope and spirit of the present invention.

Normal or fasting physiological glucose concentration thus refers to the concentration of glucose in the circulation of a subject under normal conditions (e.g., fasting basal conditions), which are distinct from transient supraphysiological, non-fasting or otherwise temporarily elevated glucose concentrations that are achieved under non-normal conditions such as after feeding or other conditions of glucose stimulation. For example by way of illustration and not limitation, depending on a variety of factors such as the physiological status, diet, activity level, health and/or genetic constitution of a subject, or the like, metabolic homeostatic mechanisms (including insulin secretion) typically operate to maintain a relatively narrow range of circulating glucose concentrations under fasting conditions that are significantly lower than circulating glucose concentrations that are reached following feeding or other glucose stimulation. Such elevated glucose concentrations, which typically are not sustained over time, reflect a departure from the normal or fasting state sought to be maintained by the homeostatic mechanisms, and are referred to herein as supraphysiological glucose concentrations. Accordingly, and as a further non-limiting example, many normal individuals may maintain a fasting or physiological circulating glucose concentration at or around approximately 40-80 mg/dl and generally less than about 110 mg/dl, which may be generally less than 126 mg/dl in an individual characterized as having “impaired fasting glucose”, and which may be generally greater than 126 mg/dl in an individual characterized as diabetic (see, e.g., Gavin et al., 2000 Diabetes Care 23 (suppl. 1):S4-S19 and references cited therein) such that a glucose concentration induced by feeding or other type of glucose stimulation that is greater than such a fasting or physiological glucose concentration in a statistically significant manner may be regarded as a supraphysiological glucose concentration. Similarly, there may be large variations among individuals with regard to circulating insulin concentrations and the degree to which an agent that impairs MCA activity according to the invention effects elevated insulin concentrations. Therefore, the present invention contemplates “enhanced” insulin secretion to refer to an insulin concentration that is, in a statistically significant manner, detectably increased by an MCA activity-impairing agent to a greater degree following supraphysiological glucose stimulation than is the degree (if any) to which the MCA activity-impairing agent increases the detectable insulin concentration under fasting or physiological conditions. Accordingly, in preferred embodiments, the agent that selectively impairs an MCA activity enhances insulin secretion that is stimulated by a supraphysiological glucose concentration and does not enhance insulin secretion in the presence of a fasting glucose concentration.

It is important to an understanding of the present invention to note that all technical and scientific terms used herein, unless otherwise defined, are intended to have the same meaning as commonly understood by one of ordinary skill in the art. The techniques employed herein are also those that are known to one of ordinary skill in the art, unless stated otherwise. Throughout this application various publications are referenced within parentheses. The disclosures of these publications in their entireties are hereby incorporated by reference in this application.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

According to certain embodiments of the present invention a “therapeutically effective amount” of an agent that impairs a MCA activity and/or an agent that lowers circulating glucose concentration may be administered. The person having ordinary skill in the art can readily and without undue experimentation determine what is a therapeutically effective amount as provided herein. Thus, for example and as described elsewhere herein, in the context of diabetes, and more specifically in the context of monitoring efficacy of diabetes therapy, periodic determination of circulating blood glucose concentrations may be routinely performed in order to determine whether a subject's blood glucose has attained a normal, physiological level. (see, e.g., Gavin et al., 2000 Diabetes Care 23 (suppl. 1):S4-S19 and references cited therein) Optionally or additionally, according to certain contemplated embodiments it may be desirable to monitor blood insulin and/or glycated hemoglobin levels, which as described herein may be performed according to any of a number of routine and well established methodologies.

Those having ordinary skill in the art are readily able to compare ATP production by an ATP biosynthetic pathway in the presence and absence of a candidate ATP biosynthesis factor. Routine determination of ATP production may be accomplished using any known method for quantitative ATP detection, for example by way of illustration and not limitation, by differential extraction from a sample optionally including chromatographic isolation; by spectrophotometry; by quantification of labeled ATP recovered from a sample contacted with a suitable form of a detectably labeled ATP precursor molecule such as, for example, ³²p; by quantification of an enzyme activity associated with ATP synthesis or degradation; or by other techniques that are known in the art. Accordingly, in certain embodiments of the present invention, the amount of ATP in a biological sample or the production of ATP (including the rate of ATP production) in a biological sample may be an indicator of altered mitochondrial function. In one embodiment, for instance, ATP may be quantified by measuring luminescence of luciferase catalyzed oxidation of D-luciferin, an ATP dependent process.

As described herein, an agent that selectively impairs MCA activity may in certain preferred embodiments interfere with transmembrane transport of calcium cations, whereby such activity may be determined by detecting calcium. A variety of calcium indicators are known in the art and are suitable for generating a detectable signal in solution or as an intracellular signal, for example, a signal that is proportional to the level of calcium in the cytosol, including but not limited to fluorescent indicators such as fura-2 (McCormack et al., 1989 Biochim. Biophys. Acta 973:420); mag-fura-2; BTC (U.S. Pat. No. 5,501,980); fluo-3, fluo-4, fluo-5F and fluo-5N (U.S. Pat. No. 5,049,673); fura-4F, fura-5F, fura-6F, and fura-FF; rhod-2, rhod-5F; Calcium Green 5N™; benzothiaza-1 and benzothiaza-2; and others, which are available from Molecular Probes, Inc., Eugene, Oreg. (see also, e.g., Calcium Signaling Protocols—Meths. In Mol. Biol.-Vol. 114), Lambert, D. (ed.), Humana Press, 1999).

Calcium Green 5N™ is a particularly preferred calcium indicator molecule for use according to the present invention. Depending, however, on the particular assay conditions to be used, a person having ordinary skill in the art can select a suitable calcium indicator from those described above or from other calcium indicators, according to the teachings herein and based on known properties (e.g., solubility, stability, etc.) of such indicators. For example by way of illustration and not limitation, whether a cell permeant or cell impermeant indicator is needed (e.g., whether a sample comprises a permeabilized cell), affinity of the indicator for calcium (e.g., dynamic working range of calcium concentrations within a sample as provided herein) and/or fluorescence spectral properties such as a calcium-dependent fluorescence excitation shift, may all be factors in the selection of a suitable calcium indicator. Calcium-Green-5N™ (potassium salt) is commercially available (Molecular Probes, Eugene, Oreg.; C-3737). Calcium-Green-5N™ is a low affinity Ca²⁺ indicator (as is, for example, Oregon Green 488 BAPTA-5N). Low affinity indicators are preferred because of the Ca²⁺ concentrations used in the assays. High affinity dyes require a lower Ca²⁺ concentration and therefore a lower number of cells, and thus a lower number of mitochondria, would be required than the number used in the assays.

Other calcium-sensitive detectable reagents that can be used in the assay of the invention include Calcein, Calcein Blue, Calcium-Green-1, Calcium-Green-2, Calcium-Green-C_{18,} Calcium Orange, Calcium-Orange-SN, Calcium Crimson, Fluo-3, Fluo-3 AM ester, Fluo-4, Fura-2, Fura-2FF, Fura Red, Fura-C₁₈, Indo-1, Bis-Fura-2, Mag-Fura-2, Mag-Fura-5, Mag-Indo-1, Magnesium Green, Quin-2, Quin-2 AM (acetoxymethyl) ester, Methoxyquin MF, Methoxyquin MF AM ester, Rhod-2, Rhod-2 AM ester, Texas Red-Calcium Green, Oregon Green 488 BAPTA-1, Oregon Green 488 BAPTA-2, BTC, BTC AM ester, (all from Molecular probes, OR), and aequorin. As noted above, in certain preferred embodiments intramitochondrial calcium concentrations are directly determined using mitochondrially targeted aequorin.

As used herein, mitochondria are comprised of “mitochondrial molecular components”, which may be a protein, polypeptide, peptide, amino acid, or derivative thereof, a lipid, fatty acid or the like, or derivative thereof; a carbohydrate, saccharide or the like or derivative thereof, a nucleic acid, nucleotide, nucleoside, purine, pyrimidine or related molecule, or derivative thereof, or the like; or another biological molecule that is a constituent of a mitochondrion. “Mitochondrial molecular components” includes but is not limited to “mitochondrial pore components”. A “mitochondrial pore component” is any mitochondrial molecular component that regulates the selective permeability characteristic of mitochondrial membranes as described above, including those that bind calcium, transport calcium or are otherwise involved in the maintenance of calcium and/or other ion levels on either side of the mitochondrial membrane. Mitochondrial pore components thus also include mitochondrial molecular components responsible for establishing calcium influx (e.g., the mitochondrial calcium uniporter) or calcium efflux (e.g., the MCA) as described herein.

Isolation and, optionally, identification and/or characterization of the MCA or any other mitochondrial molecular components with which an agent that affects intramitochondrial calcium concentration interacts may also be desirable and are within the scope of the invention. Once an agent is shown to alter a mitochondrial activity such as mitochondrial permeability properties, for example, mitochondrial binding, transport or regulation of calcium (and, optionally, sodium) cations as provided herein and in U.S. application Ser. Nos. 09/161,172, 09/338,122 and 09/434,3564, those having ordinary skill in the art will be familiar with a variety of approaches that may be routinely employed to isolate the molecular species specifically recognized by such an agent and involved in regulation of mitochondrial calcium transport, where to “isolate” as used herein refers to separation of such molecular species from the natural biological environment.

Techniques for isolating a mitochondrial molecular component such as a MCA or another mitochondrial molecular component that can artificially (e.g., pharmacologically) be influenced to maintain intramitochondrial calcium concentration, may include any biological and/or biochemical methods useful for separating the component from its biological source, and subsequent characterization may be performed according to standard biochemical and molecular biology procedures. Those familiar with the art will be able to select an appropriate method depending on the biological starting material and other factors. Such methods may include, but need not be limited to, radiolabeling or otherwise detectably labeling cellular and mitochondrial components in a biological sample, cell fractionation, density sedimentation, differential extraction, salt precipitation, ultrafiltration, gel filtration, ion-exchange chromatography, partition chromatography, hydrophobic chromatography, electrophoresis, affinity techniques or any other suitable separation method that can be adapted for use with the agent with which the mitochondrial pore component interacts. Antibodies to partially purified components may be developed according to methods known in the art and may be used to detect and/or to isolate such components. Any biological sample as provided herein may be a suitable source of biological starting material.

For example, and in certain preferred embodiments including methods for determining the presence of a MCA polypeptide in a biological sample or for isolating a MCA from a biological sample, a mitochondrial molecular component such as an MCA may be obtained from a preparation of isolated mitochondria and/or from a preparation of isolated submitochondrial particles (SMP). Techniques for isolating mitochondria and for preparing SMP are well known to the person having ordinary skill in the art and may include certain minor modifications as appropriate for the particular conditions selected (e.g., Smith, A. L., Meths. Enzymol. 10:81-86; Darley-Usman et al., (eds.), Mitochondria: A Practical Approach, IRL Press, Oxford, UK; Storrie et al., 1990 Meths. Enzymol. 182:203-255). Cell or tissue lysates, homogenates, extracts, suspensions, fractions or the like, or other preparations containing partially or fully purified mitochondrial molecular components such as mitochondrial proteins (e.g., MCA) may also be useful in these and related embodiments. According to certain other related embodiments, one or more isolated mitochondrial molecular components such as isolated MCA proteins may be present in membrane vesicles such as uni- or multilamellar membrane vesicles, or reconstituted into naturally derived or synthetic liposomes or proteoliposomes or similar membrane-bounded compartments, or the like, according to generally accepted methodologies (e.g., Jezek et al., 1990 J. Biol. Chem. 265:10522-10526).

Affinity techniques are particularly useful in the context of isolating a MCA protein or polypeptide for use according to the methods of the present invention, and may include any method that exploits a specific binding interaction involving an MCA polypeptide to effect a separation. For example, because an enzyme or an MCA polypeptide may contain covalently attached oligosaccharide moieties, an affinity technique such as binding of the MCA polypeptide to a suitable immobilized lectin under conditions that permit carbohydrate binding by the lectin may be a particularly useful affinity technique. Other useful affinity techniques include immunological techniques for isolating and/or detecting a specific MCA protein or polypeptide antigen, which techniques rely on specific binding interaction between antibody combining sites for antigen and antigenic determinants present on the factor. Binding of an antibody or other affinity reagent to an antigen is “specific” where the binding interaction involves a Ka of greater than or equal to about 10⁴ M⁻¹, preferably of greater than or equal to about 10⁵ M⁻¹, more preferably of greater than or equal to about 10⁶ M⁻¹ and still more preferably of greater than or equal to about 10⁷ M⁻¹. Affinities of binding partners or antibodies can be readily determined using conventional techniques, for example those described by Scatchard et al., Ann. N. Y Acad. Sci. 51:660 (1949).

Immunological techniques include, but need not be limited to, immunoaffinity chromatography, immunoprecipitation, solid phase immunoadsorption or other immunoaffinity methods. For these and other useful affinity techniques, see, for example, Scopes, R. K., Protein Purification: Principles and Practice, 1987, Springer-Verlag, NY; Weir, D. M., Handbook of Experimental Immunology, 1986, Blackwell Scientific, Boston; and Hermanson, G. T. et al., Immobilized Affinity Ligand Techniques, 1992, Academic Press, Inc., California; which are hereby incorporated by reference in their entireties, for details regarding techniques for isolating and characterizing complexes, including affinity techniques.

According to certain particularly preferred embodiments that relate to affinity methods as provided herein, a MCA polypeptide may be contacted with a MCA ligand under conditions and for a time sufficient to allow binding of the MCA ligand to the MCA polypeptide. Preferably, in certain embodiments the MCA ligand is immobilized on a solid-phase support as described herein. In certain other preferred embodiments, the MCA ligand is detectably labeled, as also described herein. Thus, for example, a MCA ligand may be labeled with a radionuclide such as ¹²⁵I, ¹³¹I, ³H, ¹⁴C, ⁴⁵Ca or ³⁵S, selection and labeling protocols for which are known in the art and will vary as a function of the chemical composition of the particular ligand. MCA ligands for use according to the present invention include any naturally occurring or synthetic molecule that is capable of specifically binding to a MCA as provided herein. For instance, labeled compound number 1 as described herein (Cpd. 1), or a derivative thereof as also described herein, may be covalently attached to a solid support or labeled synthetically or post-synthetically with tritium. As another example, tritiated tetraphenylphosphonium (³H-TPP), a detectably labeled derivative of the MCA inhibitor TPP (Svichar et al., 1999 NeuroReport 10:1257; Karadjov et al., 1986 Cell Calcium 7:115) may be a useful MCA ligand. Other MCA ligands that may be solid-phase immobilized or detectably labeled for use according to the subject invention methods include clonazepam and its derivatives, diltiazem and its derivatives, or other benzodiazepines and related compounds as provided herein and as known to the art (e.g., Cox and Matlib, 1993 Trends Pharmacol. Sci. 14:408; Chiesi et al., 1988 Biochem. Pharmacol. 37:4399).

In certain other embodiments, a biological sample cell may express, may be induced to express or may be transfected with a gene encoding and expressing a calcium regulatory protein such as a MCA. Calcium regulatory proteins include any naturally occurring or artificially engineered polypeptide or protein that directly or indirectly alter (e.g., increase or decrease) intracellular or intraorganellar calcium levels. Examples of calcium regulatory proteins include calmodulin, calsequestrin, calpains I and II, calpastatin, calbindin-D₉ₖ, osteocalcin, osteonectin, S-100 protein, troponin C and numerous transmembrane calcium channels. Calcium regulatory proteins also include the mitochondrial calcium uniporter. Calcium antiporter (e.g., MCA) function may play a role in a variety of normal metabolic processes, in apoptosis and in certain disease mechanisms.

For example, some transmembrane calcium channels contain functional polypeptide domains related to intracellular binding, transport or regulation of free calcium, for instance, calcium-binding, EFHAND, ion transport, ligand channel and/or calmodulin-binding IQ-domains. EFHAND, Ion Channel, Ligand Channel and IQ. For information on ion transport, see, e.g.: Williams et al., Science 257:3898-395, 1992; Jan et al., Cell 69:715-718, 1992. For information on calcium binding/transport, see, e.g.: RyRs (ryanodine receptors) Chen et al., J. Biol. Chem. 273:14675-14678, 1998. For information on L-type Ca2+ channels, see, e.g.: Hockerman et al., Annu. Rev. Pharmcol. Toxicol. 37:361-396, 1997. For information on ligand channels, see, e.g.: Tong, Science 267:1510-1512, 1995; regarding IQ, see, e.g., Xie et al., Nature 368:306-312, 1994. For information on EFHAND, see, e.g., Persechini et al., Trends Neurosci. 12:462, 1989; Ikura, Trends Biochem. Sci. 21:14, 1996; Guerini, Biochem. Biophys. Res. Commun. 235:271; Kakalis et al., FEBS Lett. 362:55, 1995. Thus, these or other calcium regulatory proteins may be expressed in a cell present in a biological sample as provided herein.

According to certain embodiments contemplated by the present invention, a cell may be a permeabilized cell, which includes a cell that has been treated in a manner that results in loss of plasma membrane selective permeability. As another example, certain calcium indicator molecules as provided herein may not be readily permeable through the plasma membrane, such that they may efficiently gain entry to the cytosol only following permeabilization of the cell. As yet another example, certain candidate agents being tested according to the method of the present invention may not be able to pass through the plasma membrane, such that a permeabilized cell provides a suitable test cell for the potential effects of such agent. Those having ordinary skill in the art are familiar with methods for permeabilizing cells, for example by way of illustration and not limitation, through the use of surfactants, detergents, phospholipids, phospholipid binding proteins, enzymes, viral membrane fusion proteins and the like; through the use of osmotically active agents; by using chemical crosslinking agents; by physicochemical methods including electroporation and the like, or by other permeabilizing methodologies.

Thus, for instance, cells may be permeabilized using any of a variety of known techniques, such as exposure to one or more detergents (e.g., digitonin, Triton X-100™, NP-40™, octyl glucoside and the like) at concentrations below those used to lyse cells and solubilize membranes (i.e., below the critical micelle concentration). Certain common transfection reagents, such as DOTAP, may also be used. ATP can also be used to permeabilize intact cells, as may be low concentrations of chemicals commonly used as fixatives (e.g., formaldehyde). Accordingly, in certain embodiments of the invention, it may be preferred to use intact cells and in certain other embodiments the use of permeabilized cells may be preferred. Other methods for permeabilizing cells include, for example by way of illustration and not limitation, through the use of surfactants, detergents, phospholipids, phospholipid binding proteins, enzymes, viral membrane fusion proteins and the like; by exposure to certain bacterial toxins, such as (α-hemolysin); by contact with hemolysins such as saponin (which is also a nonionic detergent, as is digitonin); through the use of osmotically active agents; by using chemical crosslinking agents; by physicochemical methods including electroporation and the like, or by other permeabilizing methodologies including, e.g., physical manipulations such as, e.g., electroporation. Those skilled in the art familiar with methods for permeabilizing cells will be able to determine the most appropriate permeabilizing agent based on factors including but not limited to toxicity of the agent to a specific chosen cell line, the molecular size of the agent that it is desired to have enter cells, and the like (see, e.g., Schulz, Methods Enzymol. 192:280-300, 1990).

A candidate agent for use according to the present invention, including an agent for use in a pharmaceutical composition as provided herein, may be any composition of matter that is known or suspected to be capable of selectively impairing a MCA activity as provided herein. As noted above, MCA activity according to preferred embodiments includes the calcium-sodium antiporter exchange activity described, for example, by Li et al. (1992 J. Biol. Chem. 267:17983), Cox and Matlib (1993 Trends Pharmacol. Sci. 14:408) and others with regard to a particular sodium-calcium exchange polypeptide that has been isolated and partially characterized, but the invention need not be so limited. Accordingly, MCA activity may also derive in part from one or more additional mitochondrial molecular components as provided herein that mediate calcium efflux from mitochondria subsequent to conditions (e.g., glucose stimulation) that cause transiently increased intramitochondrial calcium concentrations, such that agents of the present invention may also usefully completely or partially inhibit calcium efflux by these additional components. A variety of methods that may be employed for determining MCA activity as provided herein are known to those having ordinary skill in the art, and include by way of illustration and not limitation direct measurement of intramitochondrial calcium concentrations (e.g., by using mitochondrially targeted aequorin or other calcium indicator molecules, see Maechler et al., 1997 EMBO J. 16:3833; Kennedy et al., 1996 J. Clin. Invest. 98:2524; and references cited therein) or indirect determination of calcium released by mitochondria into the cytosol, as reported by cytosolic calcium indicator molecules such as Fura-2 or other indicators (e.g., Jung et al., 1995 J. Biol. Chem. 270:672; Li et al., 1992 J. Biol. Chem. 267:17983).

Accordingly, MCA activity may be determined by monitoring intramitochondrial and/or extramitochondrial calcium concentrations in a manner that detectably alters a signal generated by a calcium indicator molecule in a cell-based assay as described herein. Detectable alteration of a signal generated by a calcium indicator molecule typically refers to a statistically significant alteration (e.g., increase or decrease) of the signal detected at at least one of a plurality of time points. Other assays beside cell-based assays are also contemplated according to the present invention, including assays of calcium release by isolated mitochondria or of calcium uptake by submitochondrial particles (SMP) as known to the art, or assays of artificial liposomes or synthetic vesicles or the like that have been reconstituted with at least one isolated polypeptide suspected of having MCA activity as provided herein. Thus, for example, MCA activity may be detected according to any methodology whereby calcium movement across a membrane can be determined, and in particular, transmembrane calcium movement in response to an altered (e.g., increased or decreased in a statistically significant manner) sodium concentration on at least one side of the membrane. By way of illustration and not limitation, MCA activity may be detected as transmembrane calcium movement observed using a calcium-sensitive fluorescent dye (e.g., Calcium Green 5N), a calcium-sensitive electrode or a calcium-sensitive nonfluorescent dye (see, e.g., Chiesi et al., 1988 Biochem. Pharmacol. 37:4399; Rizzuto et al., 1987 Biochem. J. 246:271; Chiesi et al., 1987 Biochem. Pharmacol. 36:2735; Vaghy et al., 1982 J. Biol. Chem. 257:6000; Baysal et al., Arch. Biochem. Biophys. 291:383).

Preferably the candidate agent is provided in soluble form. Without wishing to be bound by theory, a candidate agent may directly alter the activity of a mitochondrial molecular component that regulates intramitochondrial and/or extramitochondrial free calcium levels, such as a MCA (e.g., by physical contact with the calcium channel), or may do so indirectly (e.g., by interaction with one or more additional molecular components such as mitochondrial molecular components present in a sample, where such additional components alter mitochondrial calcium regulatory activity in response to contact with the agent). Typically, and in preferred embodiments such as for high throughput screening, candidate agents are provided as “libraries” or collections of compounds, compositions or molecules. Such molecules typically include compounds known in the art as “small molecules” and having molecular weights less than 10⁵ daltons, preferably less than 104 daltons and still more preferably less than 10³ daltons.

For example, members of a library of test compounds can be administered to a plurality of samples in each of a plurality of reaction vessels in a high throughput screening array as provided herein, each containing at least one cell containing a mitochondrion (or a mitochondrion or an SMP, or an MCA-reconstituted liposome or vesicle) and a calcium indicator molecule under conditions as provided herein. The samples are contacted with a stimulus for calcium efflux (e.g., glucose in a glucose-sensitive cell; Na⁺; etc.) and then assayed for a detectable signal generated by the calcium indicator molecule at one or a plurality of time points, and the signal generated from each sample in the presence of the candidate agent is compared to the signal generated in the absence of the agent. Compounds so identified as capable of influencing mitochondrial function (e.g., alteration of MCA activity) are valuable for therapeutic and/or diagnostic purposes, since they permit treatment and/or detection of diabetes mellitus. Such compounds are also valuable in research directed to molecular signaling mechanisms that involve a MCA, and to refinements in the discovery and development of future MCA-specific compounds exhibiting greater specificity.

Candidate agents further may be provided as members of a combinatorial library, which preferably includes synthetic agents prepared according to a plurality of predetermined chemical reactions performed in a plurality of reaction vessels. For example, various starting compounds may be prepared employing one or more of solid-phase synthesis, recorded random mix methodologies and recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides (see e.g., PCT/US91/08694 and PCT/US91/04666) or other compositions that may include small molecules as provided herein (see e.g., PCT/US94/08542, EP 0774464, U.S. Pat. Nos. 5,798,035, 5,789,172, 5,751,629). Those having ordinary skill in the art will appreciate that a diverse assortment of such libraries may be prepared according to established procedures, and tested using a biological sample according to the present disclosure.

An agent so identified as one that selectively impairs (e.g., selectively decreases in a statistically significant manner) MCA function is preferably part of a pharmaceutical composition when used in the methods of the present invention. The pharmaceutical composition will include at least one of a pharmaceutically acceptable carrier, diluent or excipient, in addition to one or more selected agent that alters mitochondrial function and, optionally, other components. An agent that selectively impairs MCA activity as provided herein refers to an agent that decreases MCA function without significantly affecting other normal cellular physiologic calcium transporters. Thus, for example, an agent that selectively impairs MCA activity does not alter the activity of plasma membrane calcium channels, or of the mitochondrial calcium uniporter described above, or of other extramitochondrial calcium transport molecules.

Agents that selectively impair MCA activity as provided herein may also be useful as MCA ligands, which term includes any agent as provided herein that is capable of specific binding interactions with a mitochondrial molecular component that contributes to MCA activity, as described above. In certain embodiments the present invention thus contemplates the use of MCA ligands for a method of determining the presence of a MCA polypeptide in a sample, and in certain other embodiments the invention contemplates a method for isolating a MCA polypeptide from a biological sample according to standard affinity methodologies as described above.

Agents identified using the above assays may have remedial, therapeutic, palliative, rehabilitative, preventative and/or prophylactic effects on patients suffering from, or potentially predisposed to developing, diabetes and related diseases and disorders associated with alterations in mitochondrial function. Such diseases may be characterized by abnormal, supernormal, inefficient, ineffective or deleterious calcium regulatory activity, for example, defects in uptake, release, activity, sequestration, transport, metabolism, catabolism, synthesis, storage or processing of calcium and/or directly or indirectly calcium-dependent biological molecules and macromolecules such as proteins and peptides and their derivatives, carbohydrates and oligosaccharides and their derivatives including glycoconjugates such as glycoproteins and glycolipids, lipids, nucleic acids and cofactors including ions, mediators, precursors, catabolites and the like.

Without wishing to be bound by theory, preferred agents for diabetes may be those that lower or reduce mitochondrial calcium efflux, thereby promoting oxidative ATP synthesis in mitochondria that ultimately promotes enhanced insulin secretion. Such agents are expected to have remedial, therapeutic, palliative, rehabilitative, preventative, prophylactic or disease-impeditive effects on patients who have had, or who are thought to be predisposed to have, diabetes mellitus. For instance, a desired property of an agent that alters mitochondrial function with respect to calcium regulatory activity may be inhibition of calcium efflux from mitochondria. Accordingly, identification of agents according to the present invention that promote mitochondrial retention of calcium, e.g. by selectively impairing MCA activity, may therefore provide beneficial therapeutic agents. Similarly, in any number of other disease models, cell systems or other biological contexts, for example, in systems wherein cells are identified that are particularly sensitive to stresses from inappropriate calcium management in response to glucose stimulation (e.g., an inability to synthesize sufficient amounts of ATP capable of supporting adequate insulin secretion to return blood glucose levels to basal levels), the present invention offers opportunities to identify agents that alter aberrant mitochondrial function by altering mitochondrial calcium regulation.

“Pharmaceutically acceptable carriers” for therapeutic use are well known in the pharmaceutical art, and are described, for example, in Remingtons Pharmaceutical Sciences, Mack Publishing Co. (A. R. Gennaro edit. 1985). For example, sterile saline and phosphate-buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. Id. at 1449. In addition, antioxidants and suspending agents may be used. Id.

The pharmaceutical compositions that contain one or more agents that impair MCA activity as provided herein may be in any form which allows for the composition to be administered to a patient. For example, the composition may be in the form of a solid, liquid or gas (aerosol). Typical routes of administration include, without limitation, oral, topical, parenteral (e.g., sublingually or buccally), sublingual, rectal, vaginal, and intranasal. The term parenteral as used herein includes subcutaneous injections, intravenous, intramuscular, intrasternal, intracavernous, intrameatal, intraurethral injection or infusion techniques. The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of the invention in aerosol form may hold a plurality of dosage units.

For oral administration, which is the route of administration in preferred embodiments, an excipient and/or binder may be present. Examples are sucrose, kaolin, glycerin, starch dextrins, sodium alginate, carboxymethylcellulose and ethyl cellulose. Coloring and/or flavoring agents may be present. A coating shell may be employed.

The composition may be in the form of a liquid, e.g., an elixir, syrup, solution, emulsion or suspension. The liquid may be for oral administration or for delivery by injection, as two examples. When intended for oral administration, preferred compositions contain, in addition to one or more agents that impair MCA activity, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

A liquid pharmaceutical composition as used herein, whether in the form of a solution, suspension or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of an agent that impairs MCA activity as provided herein such that a suitable dosage will be obtained. Typically, this amount is at least 0.01 wt % of the agent in the composition. When intended for oral administration, this amount may be varied to be between 0.1 and about 70% of the weight of the composition. Preferred oral compositions contain between about 4% and about 50% of the agent(s) that alter mitochondrial function. Preferred compositions and preparations are prepared so that a parenteral dosage unit contains between 0.01 to 1% by weight of active compound.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, beeswax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the agent that impairs MCA activity of from about 0.1 to about 10% w/v (weight per unit volume).

The composition may be intended for rectal administration, in the form, e.g., of a suppository that will melt in the rectum and release the drug. The composition for rectal administration may contain an oleaginous base as a suitable nonirritating excipient. Such bases include, without limitation, lanolin, cocoa butter and polyethylene glycol. In the methods of the invention, the agent(s) that alter mitochondrial function identified as described herein may be administered through use of insert(s), bead(s), timed-release formulation(s), patch(es) or fast-release formulation(s).

It will be evident to those of ordinary skill in the art that the optimal dosage of the agent(s) that alter mitochondrial function may depend on the weight and physical condition of the patient; on the severity and longevity of the physical condition being treated; on the particular form of the active ingredient, the manner of administration and the composition employed. The use of the minimum dosage that is sufficient to provide effective therapy is usually preferred. Patients may generally be monitored for therapeutic or prophylactic effectiveness using assays suitable for the condition being treated or prevented, which will be familiar to those having ordinary skill in the art and which, as noted above, will typically involve determination of whether circulating insulin and/or glucose concentrations fall within acceptable parameters according to well known techniques. Suitable dose sizes will vary with the size, condition and metabolism of the patient, but will typically range from about 10 mL to about 500 mL for 10-60 kg individual. It is to be understood that according to certain embodiments the agent may be membrane permeable, preferably permeable through the plasma membrane and/or through mitochondrial outer and/or inner membranes. According to certain other embodiments, the use of an agent that impairs MCA activity as disclosed herein in a chemotherapeutic composition can involve such an agent being bound to another compound, for example, a monoclonal or polyclonal antibody, a protein or a liposome, which assist the delivery of said agent.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The following Examples illustrate the invention and are not intended to limit the same. Those skilled in the art will recognize, or be able to ascertain through routine experimentation, numerous equivalents to the specific substances and procedures described herein. Such equivalents are considered to be within the scope of the present invention.

### EXAMPLES

### Example 1

### Synthesis of a Representative Compound of Structure (i)

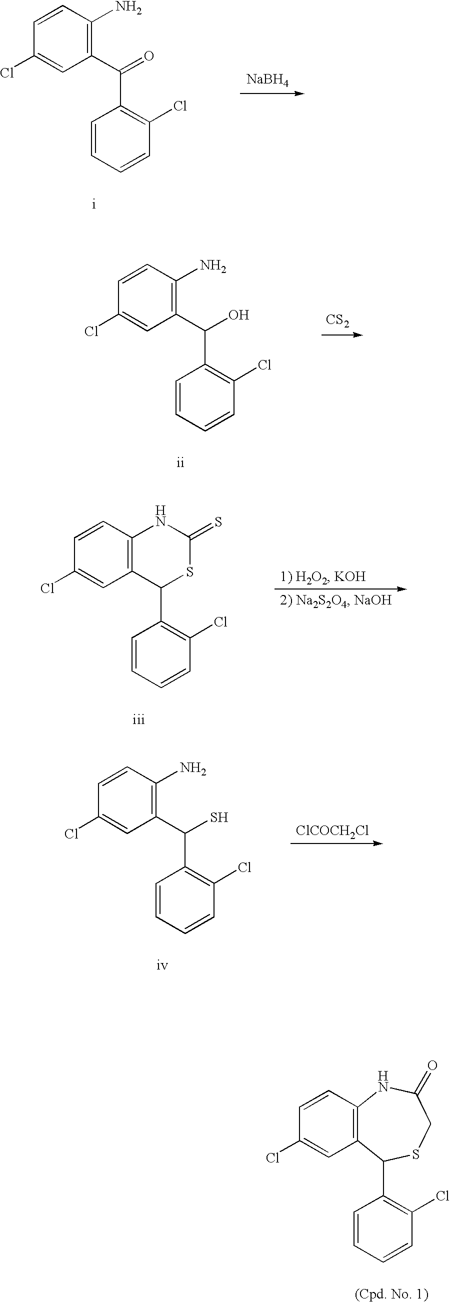

Compound No. 1 (“Cpd. No. 1”) was made according to the procedures disclosed by Hirai et al. in U.S. Pat. Nos. 4,297,280 and 4,341,704, specifically Example 3 of U.S. Pat. No. 4,297,280.

### Example 2

### Alternative Synthesis of a Representative Compound of Structure (i)

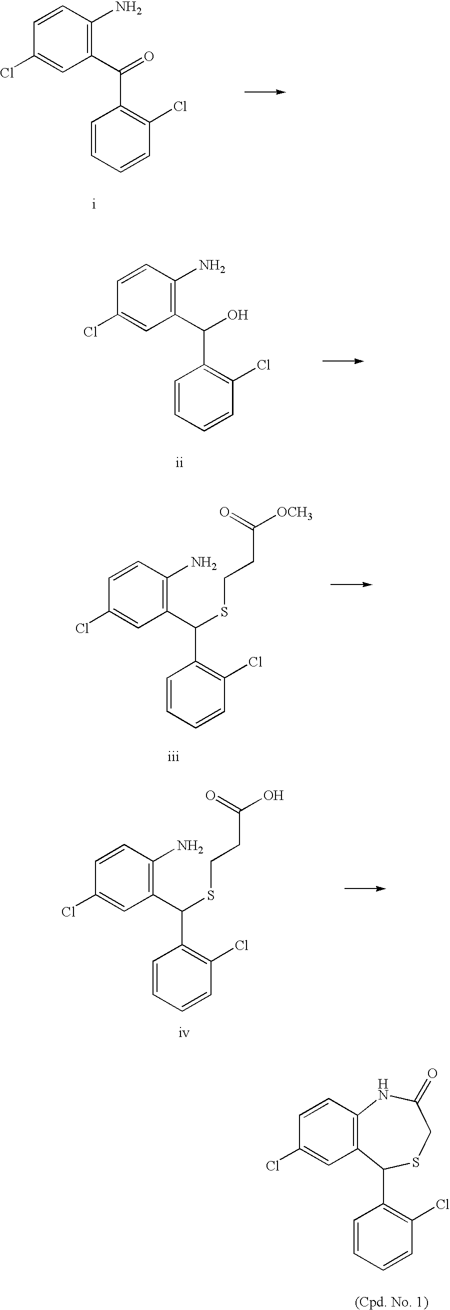

### Alcohol ii

To the starting ketone i (0.38 mmol) dissolved in THF (5 mL) under an atmosphere of nitrogen cooled to 0° C. was added lithium aluminum hydride (0.3 mL, 1.0M solution in diethyl ether). Analysis by TLC indicated the reaction was complete, saturated sodium bicarbonate (20 mL) was carefully added and the resultant solution was extracted with ethyl acetate (3×50 mL). The combined organic phase was dried over sodium sulfate, filtered and the solvent removed in vacuo to yield alcohol ii as a brown oil which was used immediately in the next step.

### Thioether iii

To a stirred solution of alcohol ii (2.00 g, 7.45 mmol) in TFA (40 mL) at room temperature was added methyl thioglycolate (2.67 mL, 29.8 mmol, 4 eq). After stirring for 96 hours, the TFA was evaporated and the residue partitioned between dichloromethane and aqueous NaOH (10%). The aqueous phase was back extracted with dichloromethane and the combined organics were dried with brine and sodium sulphate, then filtered and evaporated. The crude material (1.55 g) was adsorbed on to silica (12 g) and purified by flash chromatography on silica (120 g) with petroleum ether:ethyl acetate (5:1 then 2:1) to give thiol ether 3a (1.24 g, 3.49 mmol, 47% yield) as a pale yellow solid. (Rf(petroleum ether:ethyl acetate (5:1))=0.40). MS: calcd. for C₁₆H₁₅Cl₂NO₂S: 355.02; found: 356.0 (M+1)⁺. (Alternatively, thioether formation may be effected using 2-4 equivalents of TFA in DCM.)

### Carboxylic Acid iv

To a stirred solution of thioether iii (1.12 g, 3.15 mmol) in THF (63 mL) and methanol (63 mL) at room temperature was added sodium hydroxide solution (1 mol/L, 63 mL, 63 mmol, 20 eq). After stirring for 1 hour the solvents were evaporated and the residue partitioned between brine and dichloromethane. The aqueous phase was titrated to exactly pH 7.0 with hydrochloric acid (10%), then it was back extracted twice with further dichloromethane. The combined organics were dried with brine and sodium sulphate, then filtered and evaporated to give crude carboxylic acid iv (1.04 g, 92% crude yield) as a white solid.

### Compound No. 1

To a stirred solution of crude carboxylic acid iv (1.04 g, ˜3.03 mmol) in THF (303 mL, to give an overall concentration of ˜10 mmol/L) at room temperature was added DIEA (0.791 mL, 4.54 mmol, 1.5 eq), EDC (0.871 g, 4.54 mmol, 1.5 eq) and DMAP (37 mg, 0.30 mmol, 0.1 eq). After stirring for 20 hours the THF was evaporated then the residue was dissolved in dichloromethane and partitioned against citric acid (10%) and sodium bicarbonate (saturated aqueous solution), dried with brine and sodium sulphate, then filtered and evaporated. The crude material (2.38 g) was adsorbed onto silica (7.5 g) and purified by flash chromatography on silica (75 g) with petroleum ether:ethyl acetate (5:1 then 2:1) to give Compound No. 1 (7-chloro-5-(2-chlorophenyl)-1,5-dihydro-4,1-benzothiazepin-2-one) (0.462 g, 1.42 mol, 45% yield for two steps) as a white solid. (Rf(petroleum ether:ethyl acetate (2:1)=0.30). MS: calcd. for C₁₅H₁₁Cl₂NOS: 322.99; found: 323.8 (M+1)⁺.

### Example 3

### Synthesis of Further Representative Compounds

Using Compound No. 1 as the starting material, the following additional compounds were made:

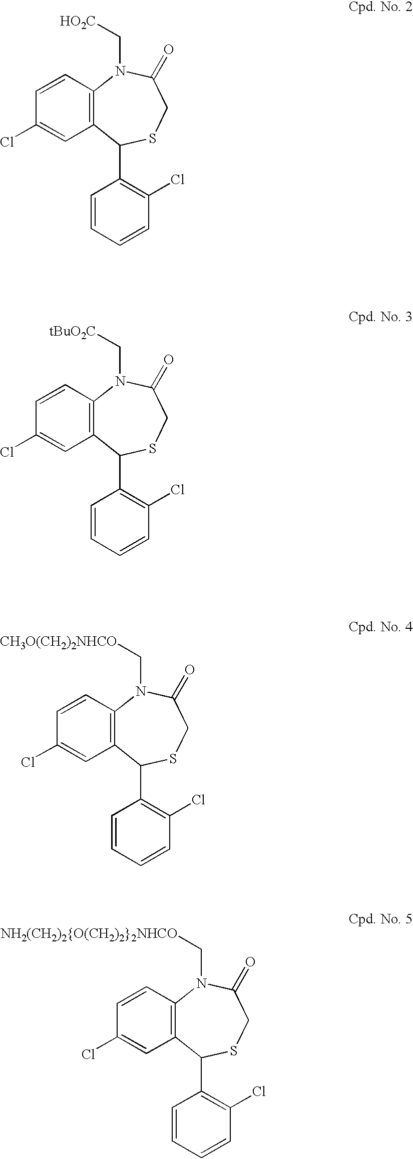

### Compound No. 3

Compound No. 1 (200 mg, 0.62 mmol) was dissolved in anhydrous tetrahydrofuran (THF, 10 ml) and added to a suspension of NaH (75 mg, 3.13 mmol) in anhydrous THF (5 ml) under a nitrogen atmosphere. The mixture was stirred at room temperature for 1 hour, following which tert-butyl bromoacetate (0.46 ml, 3.13 mmol) was added. The mixture was stirred at room temperature for additional 6 hours. The reaction was quenched with water (1 ml) and the solvent was removed under vacuum. The residue was taken up in ethyl acetate (50 ml), and washed with water (1×50 ml) and brine (1×50 ml), and dried over anhydrous sodium sulfate. The sodium sulfate was filtered off and the solvent was removed under vacuum to provide Compound No. 3. LC-MS indicated the crude product (243 mg, 89%) was analytically pure (retention time: 8.78 min; calcd. for C₂₁H₂₁Cl₂NO₃S: 437.06, found: 382.0 [(M+1)-56(t-butyl)]. ¹H NMR (CDCl₃) [symbol] 1.52 (d, 9H), 3.08 (d, 1H), 3.31 (d, 1H), 4.16 (d, 1H), 4.60 (d, 1H), 6.29 (s, 1H), 6.61 (s, 1H) 7.32(m, 2H), 7.37 (m, 1H), 7.43 (m, 2H), 7.78 (d, 1H).

### Compound No. 2

Compound No. 3 (243 mg, 0.55 mmol) was dissolved in dichloromethane (10 ml). Thioanisole (0.2 ml) and trifluoroacetic acid (10 ml) were added and the mixture was stirred at room temperature for 30 minutes. The solvent was removed by rotary evaporation and the residue was dried under high vacuum to give Compound No. 2 in quantitative yield. LC-MS indicated the crude product was analytically pure [retention time: 7.05 min; calcd. for C₁₇H₁₃Cl₂NO₃S: 381.00, found: 382.0 (M+1)]. ¹H NMR (CDCl₃)Z,900 3.13 (d, 1H), 3.32 (d, 1H), 4.36(d, 1H), 4.74 (d, 1H), 5.25 (b, 1H), 6.27 (s, 1H) 6.64 (s, 1H), 7.30 (m, 4H), 7.40 (m, 2H), 7.78 (d, 1H).

### Compound No. 4

To a solution of Compound No. 2 (10 mg, 0.026 mmol) in N,N-dimethylformamide (DMF, 1 ml) was added methoxyethylamine (0.1 ml, 1.15 mmol), diisopropylcarbodiimide (0.081 ml, 0.52 mmol) and N,N-dimethyl-4-aminopyridine (5 mg). The mixture was stirred at 60° C. for 3 days. The reaction was quenched with water (1.0 ml). The mixture was purified using RP-HPLC with a linear gradient of 5-95% acetonitrile in water in 30 minutes. The fractions were analyzed with LC-MS. The fractions containing Compound No. 4 were combined and lyophilized to give the title compound (2.7 mg, 24%). LC-MS: retention time: 6.83 minutes; calcd. for C₂₀H₂₀Cl₂N₂O₃S: 438.06, found: 438.9. ¹H NMR (CDCl₃/CD₃OD) Z,900 3.10 (d, 1H), 3.32 (d, 1H), 3.38 (s, 3H), 3.52 (m, 4H), 4.02 (d, 1H), 4.77 (d, 1H), 6.04 (s, 1H), 6.61 (s, 1), 7.31 (m, 1H), 7.37 (m, 1H), 7.42 (m, 2H), 7.48 (d, 2H), 7.76 (d, 1H).

### Compound No. 5

To a solution of Compound No 2 ( 210 mg, 0.55 mmol) in NMP (5 ml) was added N,N-diisopropylethylamine (DIEA, 0.54 ml, 1.86 mmol) and isobutyl chloroformate (0.24 ml, 1.86 mmol). The mixture was stirred at room temperature for 1 hour. 2,2′-Ethylenedioxy)bis(ethylamine) (1.8 ml, 12.4 mmol) was added. The mixture was stirred at room temperature overnight. The reaction is quenched with water (2 ml). The mixture was purified using RP-HPLC with a linear gradient of 5-95% acetonitrile in water in 30 minutes in three portions. The fractions were analyzed with LC-MS. The fractions containing Compound No. 5 were combined and lyophilized to give the title compound (67 mg, 24%). LC-MS, retention time: 5.48 minutes; calcd. for C₂₃H₂₇Cl₂N₃O₄S: 511.11, found: 512.2. ¹H NMR (CDCl₃) Z,900 3.07 (d, 1H), 3.36 (d, 1H), 3.71 (m, 6H), 3.86 (m, 6H), 4.03 (d, 1H), 4.82 (d, 1H), 6.07 (s, 1H), 6.59 (d, 1H), 7.28 (m, 1H), 7.37 (m, 1H), 7.43 (m, 1H), 7.54 (m, 1H), 7.61 (m, 1H), 7.71 (m, 1H), 7.75(d, 1H).

All LC-MS data were obtained on a ThermoQuest LCQ-deca LC/MS system (Thermoquest, San Jose, USA) under ESI conditions. The samples were eluted off a Keystone betasil C-8 column (100 mm ×2 mm, particle size 5 μm, pore size 100 Å) using a linear gradient of 5-95% acetonitrile in water in 5 min, followed by 95% acetonitrile in water for 3 min and 5% acetonitrile in water for 2 min at a flow rate of 0.3 ml/min. Both acetonitrile and water contained 0.01% TFA.

All RP-HPLC purification was carried out on a Keystone C-8 column (150 mm×20 mm, particle size 5 μm, pore size 100 Å)

### Immobilization of Compound No. 5 on Sepharose Resin.

NHS-activated sepharose resin (20 ml, 16-20 μmol/ml) was placed in a 50 ml syringe fitted with a polypropylene frit, washed with N-methypyrrolidinone (NMP, 3×30 ml) and dried by vacuum filtration. Compound No. 5 (33 mg, 64.4 μmol) was dissolved in 10% DIEA in NMP (20 ml). An aliquot (200 μl) of this solution was reserved to establish the initial time point (t=0). The remainder of the solution was added to the sepharose resin, and the slurry was gently shaken at room temperature. To monitor the progress of the reaction, a 30 μl aliquot of the reaction mixture was taken at various time points and spiked with Compound No. 1 (10 μl of a 10 mM solution) to serve as an internal standard. The mixture was diluted to 200 μl, and analyzed using LC-MS. Complete disappearance of Compound No. 5 was observed after 2.5 hrs. The resin suspension was filtered, the resin was washed with NMP (3×30 ml) and gently shaken with 20% ethanolamine in NMP (20 ml) overnight. Subsequently, the resin was washed with NMP (3×30 ml) and methanol (3×30 ml). The resin was then washed with Tris buffer (100 mM, pH 8.5, 100 ml), sodium acetate buffer (100 mM, pH 3.5, 100 ml), 20% aqueous ethanol (200 ml) and stored in 20% aqueous ethanol.

### Example 4

### Synthesis of Further Representative Compounds

By the procedures set forth in Example 2, the compounds listed in the following Table 1 were also prepared.

TABLE 1
Representative Compounds

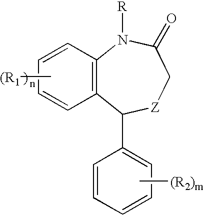

| Cpd. No. | Z | R | m | R₂ | n | R₁ |
|---|---|---|---|---|---|---|
| 6 | O | H | 1 | 2-Me | 0 | — |
| 7 | O | H | 0 | — | 0 | — |
| 8 | O | H | 0 | — | 1 | 8-Cl |
| 9 | O | H | 1 | 2-Cl | 1 | 8-Cl |
| 10 | O | H | 1 | 2-Me | 1 | 8-Cl |
| 11 | S | H | 1 | 2-Me | 1 | 8-Me |
| 12 | S | H | 2 | 2,5-diMe | 1 | 8-Cl |
| 13 | S | H | 2 | 3,5-diMe | 1 | 8-Cl |
| 14 | S | H | 2 | 3,4-diMe | 1 | 8-Cl |
| 15 | S | H | 1 | 2-Me | 0 | — |
| 16 | S | H | 1 | 3-Me | 1 | 8-Cl |
| 17 | S | H | 2 | 2,3-diMe | 1 | 8-Cl |
| 18 | S | H | 1 | 2-Me | 1 | 8-Cl |
| 19 | S | H | 2 | 2,4-diMe | 0 | — |
| 20 | S | H | 0 | — | 1 | 8-Cl |
| 21 | S | H | 2 | 2,4-diMe | 1 | 8-Cl |
| 22 | S | H | 0 | — | 1 | 8-NO₂ |
| 23 | S | H | 1 | 2-Cl | 0 | — |
| 24 | S(═O) | H | 1 | 2-Cl | 1 | 8-Cl |

Analytical data for the above representative compounds is presented in the following Table 2.

TABLE 2
Analytical Data of Representative Compounds

| Cpd. No. | MW | Formula | Calc'd | Found |
|---|---|---|---|---|
| 6 | 253.3 | C₁₆H₁₅NO₂ | 253.1 | 253.9 (M + H)⁺ |
| 7 | 239.27 | C₁₅H₁₃NO₂ | 239.1 | 240.5 (M + H) |
| 8 | 273.71 | C₁₅H₁₂ClNO₂ | 273.1 | 274.1 (M + H) |
| 9 | 308.16 | C₁₅H₁₁Cl₂NO₂ | 306.9 | 617.2 (2M + H) |
| 10 | 287.74 | C₁₆H₁₄ClNO₂ | 287.1 | 287.9 (M + H)⁺ |
| 11 | 283.39 | C₁₇H₁₇NOS | 283.1 | 284.1 (M + 1)⁺ |
| 12 | 317.83 | C₁₇H₁₆ClNOS | 317 | 318.1 (M + 1)⁺ |
| 13 | 317.83 | C₁₇H₁₆ClNOS | 317 | 318.0 (M + 1)⁺ |
| 14 | 317.83 | C₁₇H₁₆ClNOS | 317 | 318.1 (M + 1)⁺ |
| 15 | 269.36 | C₁₆H₁₅NOS | 269.1 | 270.3 (M + 1)⁺ |
| 16 | 303.81 | C₁₆H₁₄ClNOS | 303 | 304.1 (M + 1)⁺ |
| 17 | 317.83 | C₁₇H₁₆ClNOS | 317 | 318.1 (M + 1)⁺ |
| 18 | 303.81 | C₁₆H₁₄ClNOS | 303 | 607.1 (2M + 1)⁺ |
| 19 | 283.39 | C₁₇H₁₇NOS | 283 | 283.8 (M + 1)⁺ |
| 20 | 289.78 | C₁₅H₁₂ClNOS | 289 | 290.1 (M + 1)⁺ |
| 21 | 317.83 | C₁₇H₁₆ClNOS | 317 | 318.0 (M + 1)⁺ |
| 22 | 300.33 | C₁₅H₁₂N₂O₃S | 300.1 | 301.1 (M + H)⁺ |
| 23 | 289.78 | C₁₅H₁₂ClNOS | 289 | 290.1 (M + H)⁺ |
| 24 | 340.23 | C₁₅H₁₁Cl₂NO²S | 339 | 340.1 (M + H)⁺ |

### Example 5

### Mitochondrial Calcium/sodium Antiporter Inhibitor Promotes Enhanced Insulin Secretion by Insulin—Secreting Cells

INS-1 rat insulinoma cells were provided by Prof. Claes Wollheim, , Geneva, Switzerland, and cultured at 37° C. in a humidified 5% PMI cell culture media (Gibco BRL, Gaithersburg, Md.) fetal bovine serum (Irvine Scientific, Irvine, Calif.), 2 mM L-cillin, 100 U/ml streptomycin, 10 mM HEPES, 1 mM sodium pyruvate and 50 μM β-mercaptoethanol (all reagents Sigma, St. Louis, Mo., unless otherwise noted).

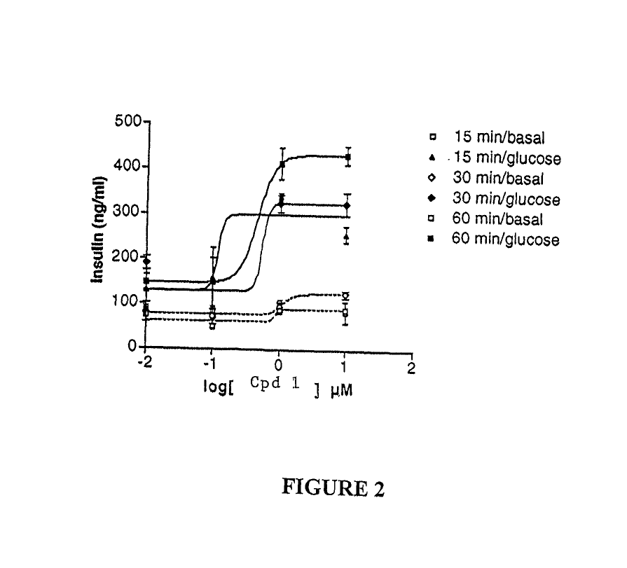

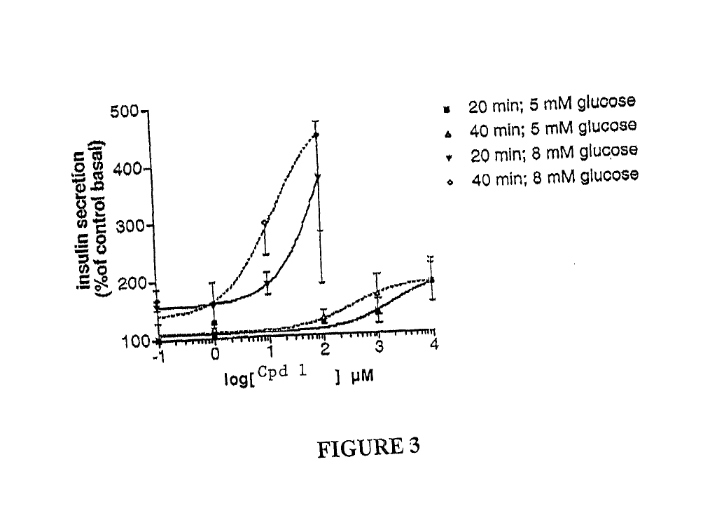

INS-1 cells were seeded into 24-well plates containing RPMI media supplemented at 0.5×10⁶ cells/well and cultured at 37° C., 5% CO₂ for 2 days. Cells at or near confluence (0.7×10⁶cells/well) were rinsed with glucose-free KRH buffer (134 mM NaCl, 4.7 mM KCl, 1.2 mM KH₂PO4, 1.2 mM MgSO₄, 1.0 mM CaCl₂, 10 mM HEPES-ph 7.4, 25 mM NaHCO₃, 0.5% BSA), then incubated in the same buffer for 1 hr at 37° C. in a humidified 5% CO₂/95% air atmosphere. Fresh KRH buffer was then added, either without added glucose (basal) or containing 8 mM glucose, in the absence or presence of the MCA inhibitor Compound No. 1 (CGP37157; 7—Chloro-5-(2-chlorophenyl)-1,5-dihydro-4,1-benzothiazepin-2(3H)-one, Tocris Cookson, Inc., Ballwin, Mo.; see, e.g., Cox et al., 1993 Trends Pharmacol. Sci. 14:408; Maechler et al. 1997 EMBO J. 16:3833; Cox et al 1993 J. Cardiovasc. Pharmacol. 21:595; White et al., 1997 J. Physiol. 498:3 1; Baron et al., 1997 Eur. J. Pharmacol. 340:295; for related compounds see, e.g., Chiesi et al., 1988 Biochem. Pharmacol. 37:4399). After an additional incubation for 15, 30 or 60 minutes at 37° C., 5% CO₂ the culture supernatants were collected. Insulin concentrations in the supernatants were measured and normalized to cell number using an insulin-specific radioimmunoassay kit (ICN Biochemicals, Irvine, Calif.) according to the manufacturer's instructions. The results are shown in FIG. 2, which illustrates enhanced glucose stimulated insulin secretion by INS-1 cells when exposed to Compound No. 1. FIG. 3 shows results that were obtained when rat pancreatic islet cells were cultured under similar conditions in the presence of “basal” (5 mM) or supraphysiological (8 mM) glucose, and in the absence or presence of various concentrations of Compound No. 1 (FIG. 3).

### Example 6

### Impairment of Transmembrane Calcium Transport by Mitochondrial Calcium/sodium Antiporter Inhibitor

Rat heart mitochondria were isolated using a modified differential centrifugation protocol essentially as described by Sordahl (Methods in Studying Cardiac Membranes (1984), pp 65-74). Experiments to assess effects on transmitochondrial membrane transport of calcium were carried out at 25° C. in a buffer consisting of 250 mM sucrose, 10 mM HEPES (pH 7.4), 5 mM Pᵢ, 5 mM succinate, 1 μM rotenone, supplemented with 1 μM cyclosporine A to prevent interference from the Ca²⁺-dependent permeability transition pore (PTP). Ca²⁺ uptake and release were measured using a Ca²+-selective electrode in a custom-constructed chamber additionally equipped for monitoring mitochondrial swelling via changes in light scattering (World Precision Instruments, Inc., Sarasota, Fla.). Alternatively, Ca²⁺ fluxes were followed fluorometrically using the Calcium Green 5N™ indicator according to the supplier's recommendations (Molecular Probes, Eugene, Oreg.).

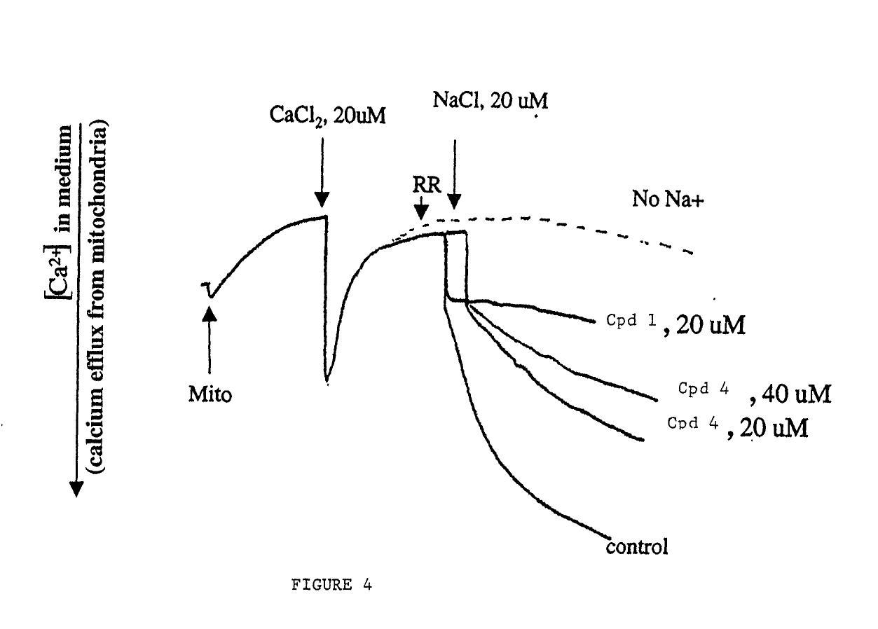

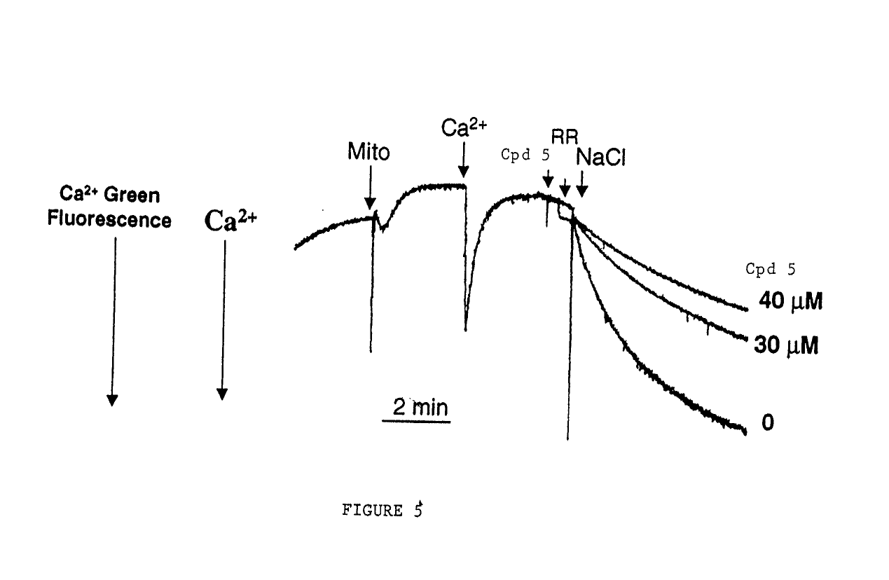

Mitochondria (0.5 mg/ml) that were energized by the presence of succinate accumulated endogenous Ca²⁺ from the medium (trace calcium from water source). Additional Ca²⁺ (20 μM ) was added to the reaction buffer 2-3 minutes following commencement of measurements with the calcium electrode. Following this Ca²⁺ loading step (Ca2+ accumulation), addition of ruthenium red (RR) was followed by a slow Ca²+efflux that according to non-limiting theory was thought to reflect Ca²⁺/H⁺ exchange (i.e., via a mechanism that involves other than the MCA). Subsequent addition of Na⁺ induced a rapid Ca²⁺ release that was inhibited completely by 20 μM Compound No. 1 (Cpd 1), a potent inhibitor of the mitochondrial Na⁺/Ca²⁺ exchanger (FIG. 4). Approximately 50% inhibition was noted with 20 μM Cpd 1. No PTP activation (swelling) was detected under experimental conditions used. FIG. 5 demonstrates Na⁺-induced Ca²⁺ release measured with Calcium Green 5N™ (0.1 μM) and its inhibition by Compound No. 5 (Cpd 5) (IC50˜30 μM). Inhibition by Compound No. 2 (Cpd 2) was observed at concentrations of 100 μM and higher.

### Example 7

### Effect of Compound No. 1 and Other Secretagogues on Insulin Secretion

As noted above, agents that selectively impair an MCA activity and other agents that may be used to treat type 2 DM include agents that enhance insulin secretion, which may also be referred to as secretagogues. This example describes effects on insulin secretion of an MCA inhibitor that is co-administered with a secretagogue.

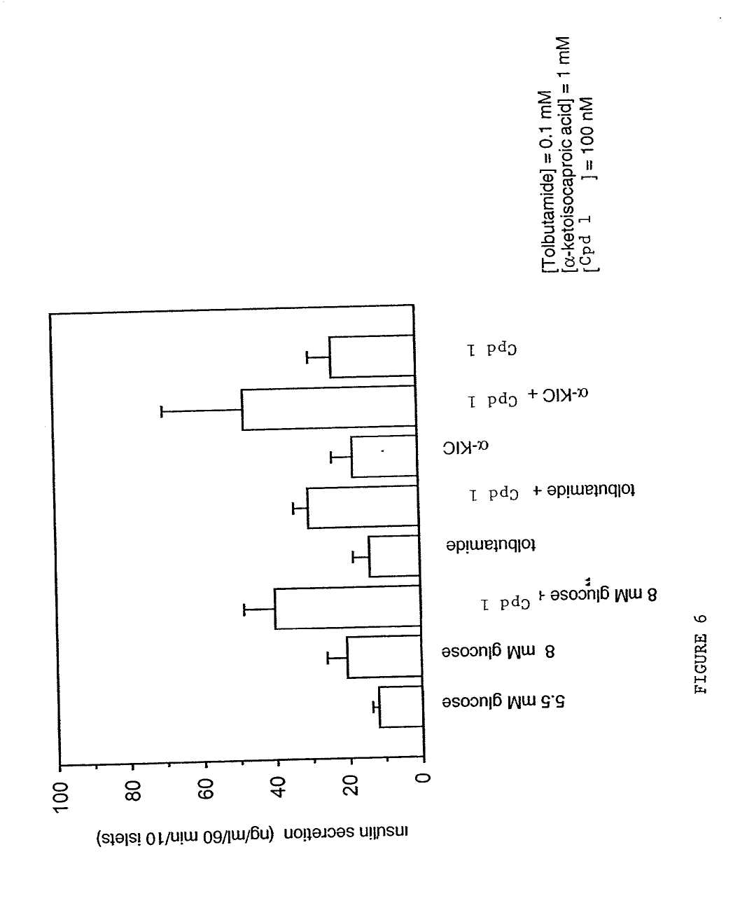

Pancreatic islets were isolated according to standard procedures. Briefly, rats were sacrificed, pancreatic ducts cannulated and the pancreases infused with 7-8 ml Hanks balanced salt solution (HBSS) containing 0.18 mg/ml collagenase (all reagents from Sigma, St. Louis, Mo. unless otherwise noted). Pancreatic tissues were then excised, minced with scissors and incubated in 10 ml of the HBSS/collagenase solution for 20 minutes at 37° C. Tissue pieces were washed twice with 1×Krebs buffer (diluted from a 5×aqueous stock containing 34.7 g/l NaCl, 1.77 g/l KCl, 0.81 g/l KH₂PO₄, 1.463 g/l MgSO₄,1.87 g/l CaCl₂, 10.4 g/l NaHCO₃, 1.35 g/l glucose, 10 g/l bovine serum albumin) and islets were manually collected, using forceps and a dissecting microscope, into clean tubes containing 1×Krebs buffer with 2 mM glucose (10 islets per tube). After a 30 minute equilibration period, test compounds were added for 40 minutes, after which media were collected for insulin determinations using a sensitive ELISA kit (Alpco, Windham, N.H.) according to the supplier's instructions. Test compound concentrations were as shown in FIG. 6: glucose (5.5 mM or 8 mM); compound 1 (Example 1, 100 nM); tolbutamide (0.1 mM); α-ketoisocaproic acid (α-KIC, 1 mM). FIG. 6 also shows the effects on insulin secretion of these compounds alone or in the indicated combinations.

### Example 8

### Use of Immobilized Compound No. 5 As Affinity Ligand for Isolation of MCA

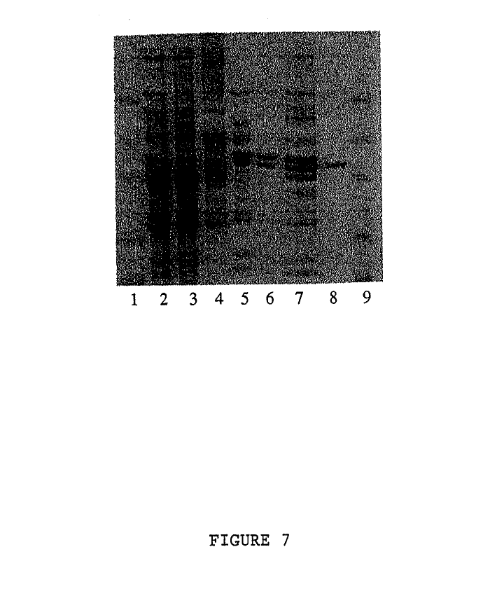

Immobilization of Compound No. 5 was described above in Example 3. Beef heart mitochondria were prepared essentially as described above and suspended in IB buffer (250 mM sucrose, 0.2 mM K+EGTA, 1 mM sodium succinate, 10 mM Tris, pH 7.8) at a protein concentration of 25 mg/ml and stored at −80° C. prior to use. To a 2 ml slurry of Compound No. 5 immobilized on NHS-activated Sepharose™ beads was added 2 ml of thawed beef heart mitochondria preparation and 5 ml of 2×column buffer (1% Triton X-100™, 2 M glycerol, 1 mM dithiothreitol, 1 mM CaCl₂, 40 mM sucrose, 1 mM TEA/EGTA and 25 mM TEA/TES, pH 7.3, supplemented with a standard protease inhibitor cocktail). The volume was brought to 10 ml by the addition of distilled water and the mixture was incubated for three hours at 4° C. with gentle agitation. The beads were pelleted by centrifugation and the supernatant was saved as the column-passed material fraction (FIG. 7, lane 3). The beads were washed twice with 2×column buffer and then packed into a disposable 10 ml column which was washed sequentially with 30 ml of 2×column buffer (FIG. 7, lane 4), 50 ml of 2×column buffer modified to contain 100 mM TEA/TES (FIG. 7, lane 5), 10 ml of the 100 mM TEA/TES buffer containing 10 mM TPP (FIG. 7, lane 6), and 50 ml of 2×column buffer containing 1 M NaCl (FIG. 7, lane 7). The column was then eluted by resuspending the beads in 10 ml of a solution containing 10 mM Cpd 1/40% (v/v) PEG 400/10% (v/v) EtOH eluate and 50% (v/v) 2×column buffer, removing the suspension to a tube and incubating the beads with gentle agitation for one hour at 4° C. The beads were pelleted and the supernatant saved; this elution step was then repeated (FIG. 7, lane 8). The collected column wash and elution fractions were standardized for protein content and electrophoresed on a 4-12% polyacrylamide-SDS NU-PAGE™ Tris-glycine gel using a MES buffering system (Invitrogen, Inc., Carlsbad, CA) according to the supplier's instructions. The gel was stained with SeeBlue Plus2™ (Invitrogen) and photographed. The results are shown in FIG. 7, including pronounced bands migrating as ˜200 kDa, ˜100 kDa and ˜50 kDa (doublet) species in the specifically (Cpd 1)-eluted material (FIG. 7, lane8).

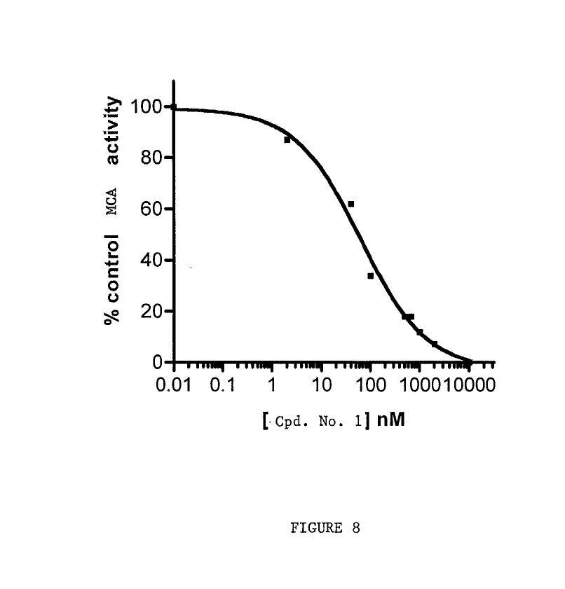

From a separate preparation of beef heart mitochondria, affinity isolation of the MCA was performed as just described, except that 1% CHAPS was substituted for 1% Triton X-100 in the initial solubilization, and following the column wash steps, retained components (including proteins bound to the affinity matrix) were eluted with 50 mM diltiazem. The column eluate containing proteins was incorporated into proteoliposomes essentially as described by Li et al (1992 J. Biol. Chem. 267: 17983-17989), using Calcium Green SN as the internal fluorescent probe (Molecular Probes, Eugene, OR), and calcium transport activity of the MCA was measured essentially as described by Li et al. (1992). Briefly, calcium chloride (100 μM) was added to a suspension of Calcium Green 5N-loaded proteoliposomes to initiate sodium-calcium exchange, which was measured as an increase in fluorescence as the calcium entered the vesicles that contained Calcium Green 5N. The effect of an MCA inhibitor, Compound No. 1 (CGP37157; 7-Chloro-5-(2-chlorophenyl)-1,5-dihydro-4,1-benzothiazepin-2(3H)-one, Tocris Cookson, Inc., Ballwin, Mo.), on sodium-calcium exchange in the proteoliposome system was tested, to confirm that affinity isolated components reconstituted into proteoliposomes possessed MCA activity. As shown in FIG. 8, sodium-calcium exchange activity was inhibited in a dose-dependent fashion by the addition of Compound No. 1. Data points in FIG. 8 represent average values at each concentration from two independent experiments.

### Example 9

### Inhibition of Mitochondrial Calcium/sodium Antiporter Activity

INS-1 rat insulinoma cells (see Example 3) were harvested by trypsinization, washed and resuspended at 10×10⁶ cells/ml in assay buffer (250 mM sucrose, 10 mM HEPES, 2.5 mM K₂HPO₄, 5 mM succinate, pH 7.4) containing 0.007% digitonin, 60 μM CaCl₂ and 0.05 μM calcium green 5N (Molecular Probes, Inc., Eugene, OR). After a five minute calcium loading incubation, ruthenium red (1 μM; Sigma, St. Louis, MO) was added to block further calcium uptake by mitochondria. Cell suspensions were dispensed into 96-well plates (100 μl per well, 1×10⁶ cells per well) and candidate agents (i.e., candidate mitochondrial calcium/sodium antiporter inhibitors) were added to some sets of triplicate wells at concentrations of 1, 10 or 100 μM, while other sets of wells provided appropriate control conditions (e.g., buffer and vehicle controls). Baseline fluorescence measurements were made using a multiwell plate fluorimeter (F-MAX™, Molecular Devices Corp., Sunnyvale, Calif.; or PolarStar , BMG Labtechnologies, Inc., Durham, N.C.) according to the manufacturer's instructions. Calcium efflux from mitochondria was then induced by adding NaCl to all wells to achieve a final concentration of 20 mM, and the rate of change in fluorescence in each well was monitored was monitored for two minutes and quantified using software included with the plate reader. Wells exhibiting significantly decreased changes in fluorescence over time relative to control wells indicated the presence of agents that were candidate MCA inhibitors, and IC₅₀ values were calculated for these compounds.

Preferred compounds of this invention have an IC₅₀ value of less than 100 μM. To this end, preferred compounds include Compound Nos. 10, 21, 23 and 24.

### Example 10

### Stimulation of Glucose-stimulated Insulin Secretion

Pancreatic islets of Langerhans were isolated from adult male Sprague-Dawley rats using a standard collagenase infusion and digestion procedure as described in Example 5. Islets were cultured at 37° C. for 1-2 days in CMRL-1066 medium supplemented with 5.5 mM glucose, 10% fetal bovine serum, 100 U/ml penicillin and 100 μg/ml streptomycin, in a humidified atmosphere containing 5% CO₂. Islets were manually picked and washed in Krebs Ringer Bicarbonate buffer (KRB: 134 mM NaCl, 4.7 mM KCl, 1.2 mM KH₂PO₄, 1.2 mM MgSO₄, 1 mM CaCl₂, 10 mM NaHCO₃, pH 7.4) in preparation for measurement of glucose-stimulated insulin secretion (GSIS). Aliquots of washed islets were preincubated in oxygenated KRB supplemented with 16 mM HEPES, 0.01% fetal bovine serum and 5.5 mM glucose for 60 min at 37° C. Compounds to be tested (e.g., candidate mitochondrial calcium/sodium antiporter inhibitors) were added at various concentrations for 10 minutes, after which additional glucose was added to different islet cultures to achieve a final glucose concentration of 5.5, 8, 11 or 20 mM, and incubations were allowed to proceed an additional 20 min. Cell-conditioned media samples were then collected by centrifugation and their insulin content was determined using enzyme-linked immunosorbent assay (ELISA) kits (CrystalChem or ALPCO rat insulin ELISA) according to the kit supplier's instructions. Following treatment with a preferred compound, the concentration of insulin detected in the islet-conditioned medium was at least 1.5 times the insulin concentration detected in the medium conditioned by islets that were exposed to 8 mM glucose. At a concentration of 1 μM, CGP37157 (7-Chloro-5-(2-chlorophenyl)-1,5-dihydro-4,1-benzothiazepin-2(3H)-one, Tocris Cookson, Inc., Ballwin, Mo.) stimulated islet GSIS by 222 (±48) % relative to GSIS detected with 8 mM glucose; at the same concentration (1 μM), Compound No. 24 stimulated GSIS by 178% and Compound No. 18 stimulated GSIS by 165%.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for treating diabetes mellitus, comprising:
administering, to a subject having or suspected of being at risk for having diabetes mellitus, a therapeutically effective amount of a pharmaceutical composition comprising an agent that selectively impairs a mitochondrial calcium/sodium antiporter activity.

2. A method for treating diabetes mellitus, comprising:
administering, to a subject having or suspected of being at risk for having diabetes mellitus, a therapeutically effective amount of a pharmaceutical composition comprising an agent that selectively impairs a mitochondrial calcium/sodium antiporter activity wherein said agent enhances insulin secretion.

3. A method for treating diabetes mellitus, comprising:
administering, to a subject having or suspected of being at risk for having diabetes mellitus, a therapeutically effective amount of a pharmaceutical composition comprising an agent that selectively impairs a mitochondrial calcium/sodium antiporter activity wherein said agent enhances insulin secretion that is stimulated by glucose.

4. A method for treating diabetes mellitus, comprising:
administering, to a subject having or suspected of being at risk for having diabetes mellitus, a therapeutically effective amount of a pharmaceutical composition comprising an agent that selectively impairs a mitochondrial calcium/sodium antiporter activity wherein said agent enhances insulin secretion that is stimulated by a supraphysiological glucose concentration and does not enhance insulin secretion in the presence of a fasting physiological glucose concentration.

5. The method of any one of claims 1-4 wherein the diabetes mellitus is type 2 diabetes mellitus.

6. The method of any one of claims 1-4 wherein the diabetes mellitus is maturity onset diabetes of the young.

7. The method of any one of claims 1-4 wherein the pharmaceutical composition is administered orally.

8. The method of any one of claims 1-4 wherein the agent does not substantially alter insulin secretion in the presence of a fasting physiological glucose concentration.

9. The method of any one of claims 1-4 wherein the candidate agent is membrane permeable.

10. The method of claim 9 wherein the membrane is at least one of the membranes selected from the group consisting of a plasma membrane and a mitochondrial membrane.

11. The method of claim 10 wherein the mitochondrial membrane is selected from the group consisting of an inner mitochondrial membrane and an outer mitochondrial membrane.

12. A method for determining the presence of a mitochondrial calcium/sodium antiporter polypeptide in a biological sample comprising:
contacting a biological sample containing a mitochondrial calcium/sodium antiporter polypeptide with a mitochondrial calcium/sodium antiporter ligand under conditions and for a time sufficient to allow binding of the mitochondrial calcium/sodium antiporter ligand to a mitochondrial calcium/sodium antiporter polypeptide; and
detecting the binding of the mitochondrial calcium/sodium antiporter ligand to a mitochondrial calcium/sodium antiporter polypeptide, and therefrom determining the presence of a mitochondrial calcium/sodium antiporter polypeptide in said biological sample.

13. The method of claim 12 wherein the mitochondrial calcium/sodium antiporter ligand comprises Compound No. 1 or a derivative thereof.

14. The method of claim 12 wherein the mitochondrial calcium/sodium antiporter ligand is detectably labeled.

15. The method of claim 14 wherein the detectably labeled mitochondrial calcium/sodium antiporter ligand comprises a radiolabeled substituent.

16. The method of claim 15 wherein the radiolabeled substituent is selected from the group consisting of ¹²⁵I, ¹³¹I, ³H, ¹⁴C, ⁴⁵Ca and ³⁵S.

17. The method of claim 12 wherein the detectably labeled mitochondrial calcium/sodium antiporter ligand comprises a fluorescent substituent.

18. The method of claim 14 wherein the detectable detectably labeled mitochondrial calcium/sodium antiporter ligand comprises covalently bound biotin.

19. A method for isolating a mitochondrial calcium/sodium antiporter from a biological sample, comprising:
contacting a biological sample suspected of containing a mitochondrial calcium/sodium antiporter polypeptide with a mitochondrial calcium/sodium antiporter ligand under conditions and for a time sufficient to allow binding of the mitochondrial calcium/sodium antiporter ligand to a mitochondrial calcium/sodium antiporter polypeptide; and
recovering the mitochondrial calcium/sodium antiporter polypeptide, and thereby isolating a mitochondrial calcium/sodium antiporter from a biological sample.

20. The method of claim 19 wherein the mitochondrial calcium/sodium antiporter ligand is covalently bound to a solid phase.

21. The method of claim 19 wherein the mitochondrial calcium/sodium antiporter ligand is non-covalently bound to a solid phase.

22. The method of any one of claims 1-4 further comprising administering to the subject one or more agent that lowers circulating glucose concentration in the subject.

23. The method of claim 22 wherein the agent that lowers circulating glucose concentration is selected from the group consisting of insulin, an insulin secretagogue, an insulin sensitizer, an inhibitor of hepatic glucose output and an agent that impairs glucose absorption.

24. The method of claim 23 wherein the insulin secretagogue is selected from the group consisting of a sulfonylurea compound and a nonsulfonylurea compound.

25. The method of any one of claims 1-4 wherein the agent has the following structure: 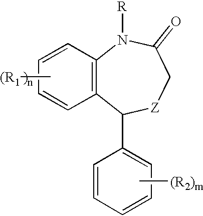
or a stereoisomer, prodrug or pharmaceutically acceptable salt thereof,
wherein
Z is O, S, S(═O) or S(═O)₂;
R is hydrogen, alkyl or substituted alkyl;
R₁ and R₂ are the same or different and at each occurrence are independently halogen, cyano, nitro, mono- or di-alkylamino, alkyl, substituted alkyl, aryl, substituted aryl, arylalkyl, substituted arylalkyl, heterocycle, substituted heterocycle, heterocyclealkyl or substituted heterocyclealkyl; and
n and m are the same or different, and independently 0, 1, 2, 3 or 4.

26. The method of claim 25 wherein Z is oxygen.

27. The method of claim 25 wherein Z is sulfur.

28. The method of claim 25 wherein n is 1.

29. The method of claim 25 wherein m is 1.

30. The method of claim 28 wherein R₁ is halogen.

31. The method of claim 29 wherein R₂ is halogen.

32. The method of claim 30 wherein R₁ is halogen at the 8-position.

33. The method of claim 31 wherein R₂ is halogen at the 2-position.

34. The method of claim 25 wherein R is hydrogen.

35. The method of claim 25 wherein R is substituted alkyl.

36. The method of claim 35 wherein alkyl is substituted with —C(═O)ORₐ.

37. The method of claim 36 wherein Rₐ is hydrogen or alkyl.

38. The method of claim 35 wherein alkyl is substituted with —CONRₐ{alkanediyl)OR_{b} or —CONR_{c}{alkanediyl-O)₁₋₆(alkanediyl)NRₐR_{b},.

39. The method of claim 3 8 wherein —CONRₐ{alkanediyl)OR_{b} is —CONH(CH₂)₂OCH₃.

40. The method of claim 38 wherein —CONR_{c}{alkanediyl-O)₁₋₆(alkanediyl)NRₐR_{b} is —CONH{(CH₂)₂O}₂(CH₂)₂NH₂.
